SEQUENCE LISTING (25 sequences)

SEQ ID NO: 1 - protein, 380 aa, synthetic construct
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRHHHHHH

SEQ ID NO: 2 - protein, 246 aa, synthetic construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKR

SEQ ID NO: 3 - protein, 128 aa, synthetic construct
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 4 - protein, 385 aa, synthetic construct
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRHHHHHH

SEQ ID NO: 5 - protein, 133 aa, synthetic construct
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 6 - protein, 243 aa, synthetic construct
QVNLLQSGAALVKPGASVKLSCKASGYTFTDFYIHWVKQSHGKSLEWIGYINPNSGYTNYNEKFKNKATLTVDKSTSTGYMELSRLTSEDSANYSCTRGVPGNNWFPYWGQGTLVTVSSGGGGSGGGGSGGGGSALDTVLTQPPALTVSPGEKLTISCKASESVTSRMHWYQQKPGQQPKLLIYKASNLASGVPARFSGSGSGTDFTLTIDPVEADDTAIYFCQQSWNGPLTFGAGTKLELKR

SEQ ID NO: 7 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 8 - protein, 10 aa, synthetic construct
GGGGSGGGGS

SEQ ID NO: 9 - protein, 20 aa, synthetic construct
GGGGSGGGGSGGGGSGGGGS

SEQ ID NO: 10 - protein, 30 aa, synthetic construct
GGGGSGSGGGSGGGGSGSGGGGSGSGGGGS

SEQ ID NO: 11 - skipped

SEQ ID NO: 12 - protein, 126 aa, synthetic construct
EVQLVESGGGLVQPGGSLRLSCAASGFTLDYLAIGWFRQAPGKEREGVSCVSSSGQYTYYADSVKGRFTISRDNAESTVYLQMNSLKPEDTAVYYCATDPECYRVRGYYNGEYDYWGQGTQVTVSS

SEQ ID NO: 13 - protein, 123 aa, synthetic construct
EVQLVESGGGLVQAGGSLRLSCAASGRTFSWSAVGWFRQAPGKEREFVAAIRWSGGSPYYADSVKDRFTISRDNAKNTVYLQMNSLRPEDTAVYLCGETSLFPTSRGSHYDTWGQGTQVTVSS

SEQ ID NO: 14 - protein, 166 aa, synthetic construct
SPGQGTQSENSCTHFPGNLPAALRALRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 15 - protein, 123 aa, synthetic construct
QVQLQESGGGSVQTGGSLRLSCAASQVNMNSVGWFRQAPGKAREGVATISPSGGATYYTDSVKGRFTISRDNAKNTVYLQMNSLNAEDTAMYYCAADGGRDWWLLRPQTFSYWGQGTQVTVSS

SEQ ID NO: 16 - protein, 347 aa, synthetic construct
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSGGGGSHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 17 - protein, 352 aa, synthetic construct
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGGGSHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 18 - protein, 254 aa, synthetic construct
QVQLQESGGGSVQTGGSLRLSCAASQVNMNSVGWFRQAPGKAREGVATISPSGGATYYTDSVKGRFTISRDNAKNTVYLQMNSLNAEDTAMYYCAADGGRDWWLLRPQTFSYWGQGTQVTVSSGGSEVQLVESGGGLVQAGGSLRLSCAASGRTFSWSAVGWFRQAPGKEREFVAAIRWSGGSPYYADSVKDRFTISRDNAKNTVYLQMNSLRPEDTAVYLCGETSLFPTSRGSHYDTWGQGTQVTVSSGAPGS

SEQ ID NO: 19 - protein, 295 aa, synthetic construct
EVQLVESGGGLVQPGGSLRLSCAASGFTLDYLAIGWFRQAPGKEREGVSCVSSSGQYTYYADSVKGRFTISRDNAESTVYLQMNSLKPEDTAVYYCATDPECYRVRGYYNGEYDYWGQGTQVTVSSGGSSPGQGTQSENSCTHFPGNLPAALRALRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 20 - protein, 7 aa, Synthetic construct
GGGGSSS

SEQ ID NO: 21 - protein, 12 aa, synthetic construct
SCVLMRCGGCCN

SEQ ID NO: 22 - protein, 14 aa, synthetic construct
PSCVPLMRCGGCCN

SEQ ID NO: 23 - protein, 9 aa, synthetic construct
GDLIYRNQK

SEQ ID NO: 24 - protein, 23 aa, synthetic construct
GGGGGGGGGPSCVOLMRCGGCCN

SEQ ID NO: 25 - protein, 14 aa, synthetic construct
GSTSGSGKSSEGKG

(19) United States
(12) Patent Application Publication
(10) Pub. No.: US 2025/0250329 A1
(43) Pub. Date: Aug. 7, 2025

(54) CYTOKINE ADAPTOR PROTEINS AND USES THEREOF

(71) Applicant: The Board of Trustees of the Leland Stanford Junior University, Stanford, CA (US)

(72) Inventors: Kenan Christopher Garcia, Menlo Park, CA (US); Caleb Glassman, Redwood City, CA (US); Robert Saxton, Palo Alto, CA (US); Gita Chu Abhiraman, Stanford, CA (US); Karsten D. Householder, Scottsdale, AZ (US)

(21) Appl. No.: 18/855,249

(22) PCT Filed: Apr. 10, 2023
(86) PCT No.: PCT/US2023/065596

§ 371 Date: Oct. 8, 2024

Related U.S. Application Data
(60) Provisional application No. 63/329,773, filed on Apr. 11, 2022.

(51) Int. Cl. C07K 16/22; C07K 16/28

(52) U.S. Cl. CPC C07K 16/22; C07K 16/2866; C07K 2317/569; C07K 2317/622; C07K 2319/21

(57) ABSTRACT

Cytokine adaptors, compositions comprising cytokine adaptors, and methods of use thereof, are provided. Cytokine adaptors are bi-specific binding proteins that alter the specificity of a receptor/ligand interaction. The adaptors generally act by simultaneously binding to a target protein and a receptor to drive a context-dependent response.

### CROSS REFERENCE TO OTHER APPLICATIONS

This application claims the benefit of U.S. Provisional Application No. 63/329,773, filed Apr. 11, 2022, the contents of which are hereby incorporated by reference in its entirety. INCORPORATION BY REFERENCE LISTING PROVIDED AS A TEXT

A sequence listing is provided herewith as a sequence listing xml, “S22-118_STAN-1905WO_SeqList” created on Apr. 10, 2023, and having a size of 25,304 Bytes. The contents of the sequence listing xml are incorporated by reference herein in their entirety.

### BACKGROUND

Cytokine signaling is essential to maintaining homeostasis in health and disease. Cytokines can become dysregulated in disease, leading to excessive immune suppression or inflammation. Cytokine antagonists have emerged as recent therapies especially in the treatment of autoimmune disease, however, cytokine receptor agonism has been difficult to leverage clinically due to the pleiotropy and the broad expression of cytokine receptors in off-target tissues. New methods of context-dependent cytokine agonism, paired with cytokine antagonism, could be useful in the treatment of a wide range of diseases including cancer and autoimmune disease.

A tumor microenvironment (TME) may be broadly defined as the non-transformed cells, molecules, and blood vessels that surround and feed the transformed tumor cells. A tumor can change its microenvironment, and the microenvironment can affect how a tumor grows and spreads. Central to this activity is the presence of secreted factors, including secreted proteins, that can act on non-transformed cells and alter their behavior. Aspects of the TME include the presence of factors that support cancer cell growth and promote immune cell tolerance.

Although cytotoxic immune effector cells can be recruited to a tumor site, their anti-tumor functions are often downregulated in response to factors present in the TME. For example, infiltrates of inflammatory cells present in human tumors can be exhausted or anergic in nature, and enriched in regulatory T cells (T_{reg}) as well as myeloid suppressor cells (MSC). Immune cells in the tumor microenvironment may not only fall to exercise anti-tumor effector functions, but can be co-opted to promote tumor growth.

Transforming growth factor β (TGF-β) signaling plays an important role in tumor initiation and progression. TGF-β is often produced in large quantities by tumor cells and is known to be pro-oncogenic in late stages of cancer. The mechanisms of TGF-β tumor promotion include dysregulation of cyclin-dependent kinase inhibitors, alteration in cytoskeletal architecture, increases in proteases and extracellular matrix formation, decreased immune surveillance and increased angiogenesis. Molecules such as TGF-β, IL-10, and VEGF are produced by tumors, and have been targeted by therapeutic antibodies to treat cancer.

The result of activation of one or several molecular mechanisms that lead to inhibition of immune cell functions or to apoptosis of anti-tumor effector cells is tumor escape from the host immune system. The ability to therapeutically block tumor escape depends on understanding and manipulation of cellular and molecular pathways operating in the tumor microenvironment. Novel therapeutic strategies that change the pro-tumor microenvironment to one favoring anti-tumor activity is of great interest and addressed herein.

In autoimmune diseases pro-inflammatory cytokines, including IL-23, IL-17, and TNF-α, are often produced in large quantities. The imbalance between pro-inflammatory cytokine production and immunosuppressive cytokine production contributes to disease pathology in autoimmune conditions including inflammatory bowel disease (IBD), psoriasis, rheumatoid arthritis (RA). Blockade of IL-23, TNF-α, and IL-17, have been used therapeutically for the treatment of autoimmune disease. Restoring homeostatic balance in inflamed tissues remains a challenge in the treatment of autoimmune disease. Novel therapeutic strategies that change the pro-inflammatory environment to a tolerogenic environment are addressed within.

### SUMMARY

Cytokine adaptors, compositions comprising cytokine adaptors, and methods of use thereof, are provided. Cytokine adaptors are bi-specific binding proteins that alter the specificity, e.g. the signaling pathway that is activated, of a receptor/ligand interaction. The adaptors can act by binding to a tumor-associated protein, e.g. an immunosuppressive cytokine, and a pro-inflammatory receptor, to convert a suppressive signal into an activating stimulus. Alternatively, an adaptor can bind to a pro-inflammatory cytokine, e.g. one associated with an autoimmune disease, and convert the signal to an immunosuppressive signal. Cytokine adaptors can be administered for one or more of: preventing, inhibiting, and treating a cytokine associated disease, e.g. cancer associated with immunosuppressive cytokines in an individual in need thereof; or inflammatory conditions associated with proinflammatory cytokines, e.g. autoimmunity, graft rejection, asthma, and the like.

A cytokine adapter protein comprises a first binding domain (which may be referred to as a target binding domain) that specifically binds to a target protein, which can be a tumor-associated protein, e.g. a suppressive cytokine; or a proinflammatory cytokine, where the target protein is a secreted protein or a surface protein that exists as a monomer, dimer, or a multicomponent protein complex. In some embodiments the target protein is an immunosuppressive cytokine present in the TME that decreases immune responsiveness to tumor cells, including without limitation TGF-β, IL-10, etc., or a factor that enhances tumor growth and/or metastasis, e.g. VEGF, etc. A tumor-associated protein can be synthesized by tumor cells, or can be synthesized by non-transformed cells present in the TME. In some embodiments the target protein is a proinflammatory cytokine, e.g. an IL-17 family member; IL-23, IL-1β, IL-6, TNF-α, etc.

A cytokine adapter protein also comprises a second binding domain (which may be referred to as a signal activator binding domain) that specifically binds to one component of a multicomponent signal receptor. In some embodiments, e.g. paired with a suppressive cytokine, the signal receptor is a proinflammatory signal receptor, including without limitation receptors such as IL-2 receptor beta (IL-2β, CD122). IL-2R gamma. (IL-2Rγ, γc, CD132), etc. In some embodiments, the pro-inflammatory signal receptor is expressed by immune cells present in the TME and increases immune responsiveness to tumor cells when activated. In other embodiments, e.g. paired with a proinflammatory cytokine, the signal receptor is a suppressive cytokine receptor, e.g. IL-10Rα and IL-10Rβ, and IL-12Rβ1; etc.

In one embodiment, cytokine adaptors are administered as non-identical pairs, wherein each of the target binding domains of one pair member can (a) bind to the same epitope on the tumor-associated protein in the case of a homodimer; or (b) bind to two different epitopes of the same tumor-associated protein in the case of a monomer, or (c) bind to two different tumor-associated proteins in a multiprotein complex, e.g. a heterodimer, heterotrimer, etc. Each of the signal activator binding domains in a pair of cytokine adaptors binds to different proteins of a proinflammatory signal receptor, where the different proteins activate signaling when multimerized, e.g. one cytokine adaptor binds to IL-2Rβ and the second cytokine adaptor binds to IL-2Rγ; or one cytokine adaptor binds to IL-10Rα and the second cytokine adaptor binds to IL-10Rβ.

In one embodiment, where the target protein forms a homodimer, the first cytokine adaptor in the pair binds to a first target protein present in the homodimer and the second cytokine adaptor in the pair binds to the same epitope on a second target protein present in the homodimer, thereby bringing the signal activator binding domains into close proximity. The target signal activator binding domains can then bind to each of the different proteins of the signal receptor, thereby activating signal transduction. In some embodiments, the first and second cytokine adaptors in a pair do not activate the signal receptor in the absence of the target protein.

In another embodiment, the target protein is a monomer, where the first cytokine adaptor in the pair binds to a first epitope on the target protein, and the second cytokine adaptor in the pair binds to a different, non-overlapping, epitope on the target protein. The target signal activator binding domains can then bind to each of the different proteins of the signal receptor, thereby activating signal transduction. In some embodiments, the first and second cytokine adaptors in a pair do not activate the signal receptor in the absence of the target protein.

In one embodiment, the target proteins form a heterodimer or multimer, where the first cytokine adaptor in a pair binds to an epitope on a first target protein and the second cytokine adaptor in a pair binds an epitope on a second target protein, thereby bringing the signal activator domains into close proximity. The target signal activator binding domains can then bind to each of the different proteins of the signal receptor, thereby activating signal transduction.

In one embodiment, the target protein is a monomeric protein, where the first cytokine adaptor in a pair binds to a first epitope on the target protein and the second cytokine adaptor in a pair binds to a second epitope on the target protein, where the first and second epitopes are positioned to bring the signal activator domains into close proximity upon binding. The target signal activator binding domains can then bind to each of the different proteins of the signal receptor, thereby activating signal transduction.

In some embodiments, binding of a cytokine adaptor to the target protein inactivates the target protein. In other embodiments, binding of a cytokine adaptor to the target protein does not inactivate the target protein.

The first and second binding domains of cytokine adaptor proteins can comprise binding domains of any type. In some embodiments, a target binding domain comprises, without limitation, a nanobody, a DARPin (designed ankyrin repeat protein), an antibody, an antibody fragment, a heavy chain only antibody, a single-chain variable fragment (scFv), an immunoglobulin single variable domain (ISV), etc. In some embodiments, the first and the second binding domains are of the same type, e.g. a first binding domain comprising an ISV and a second signal activator binding domain comprising an ISV. In some embodiments, the first and second binding domains are of a different type, e.g. a first binding domain comprising a scFv and a second signal activator binding domain comprising an ISV or vice versa.

The first and second binding domains may be contiguous within one domain, or separated by a linker, e.g. a polypeptide linker, or a non-peptidic linker, etc. The length of the linker, and therefore the spacing between the binding domains, can be used to modulate the signal strength, and can be selected depending on the desired use of the cytokine adaptor. The enforced distance between binding domains can vary, but in certain embodiments may be less than about 100 angstroms, less than about 90 angstroms, less than about 80 angstroms, less than about 70 angstroms, less than about 60 angstroms, or less than about 50 angstroms.

In some embodiments the linker is a rigid linker, in other embodiments the linker is a flexible linker. Where the linker is a peptide linker, it may be from about 1, 2, 3, 4, 5, 6, 7, 8, 9, 10, 11, 12, 13, 14, 15, 16, 17, 18, 19, 20 21, 22, 23, 24, 25, 26, 27, 28, 29, 30 or more amino acids in length, and is of sufficient length and amino acid composition to enforce the distance between binding domains. In some embodiments the linker comprises or consists of one or more glycine and/or serine residues.

Target binding domains can be specific for any target protein of interest, secreted or surface expressed, including without limitation immunosuppressive cytokines; or proinflammatory cytokines For example, tumor-associated target proteins include, without limitation, TGF-β, IL-10, IL-23, vascular endothelial growth factor (VEGF), programmed death-ligand 1 (PDL1), human epidermal growth factor 2 (HER2), epidermal growth factor receptor (EGFR), fbroblast activation protein (FAP), tumor-associated calcium signal transducer 2 (Trop2), epithelial cell adhesion molecule (EPCAM), prostate-specific membrane antigen (PSMA), arginase 2 (ARG2), etc. In some embodiments, a tumor-associated protein of interest is selected based on concentration or expression in tumor tissues. For example, tumor-associated proteins may be found in other areas outside of the TME but are generally present at a relatively high concentration in the TME, e.g. a TME concentration greater than 2× the serum concentration, greater than 5× the serum concentration, greater than 10× the serum concentration, greater than 20× the serum concentration, or more. When tumor-associated proteins are found outside of the TME, the dosing of cytokine adaptors can be adjusted such that the cytokine adaptors only produce pro-inflammatory signals in the local tumor environment, and not at distal sites.

Inflammation-associated target proteins of interest include, without limitation, proinflammatory cytokines, including IL-23, IL-17A, IL-17B, IL-17C, IL-17C, IL-17D, IL-17E, IL-17F, IL-1β, IL-6, TNF-α, etc. Inflammation associated target proteins may, for example, be present at a relatively high concentration in inflammatory lesions, such as psoriasis lesions, the CSF of multiple sclerosis, synovial fluid of rheumatoid arthritis, and the like, a lesion concentration may be greater than 2× the serum concentration, greater than 5× the serum concentration, greater than 10× the serum concentration, greater than 20× the serum concentration, or more. When proinflammatory cytokines are present outside of lesions, the dosing of cytokine adaptors can be adjusted such that the cytokine adaptors only produce suppressive signals in the local environment, and not at distal sites.

Target signal activator binding domains may bind any two-component signaling complex of interest. Two-component signaling complexes of interest include, without limitation, IL-2Rβ and IL-2Rγ, interferon-α/β receptor 1 (IFNAR1), interferon-α/β receptor 2 (IFNAR2), IL-10Rα and IL-10Rβ, IL23R and IL-12Rβ1; etc.

In some embodiments a pair of cytokine adaptors each specifically bind to an epitope of TGF-β as a target binding protein, where the epitope recognized by each adaptor can be the same or different. In some embodiments the target signal activator proteins recognized by the pair of adaptors are IL-2Rβ (CD122), and IL-2Rγ (CD132), respectively. In some embodiments one or more of the binding domains are selected from a nanobody, a DARPin (designed ankyrin repeat protein), an antibody, an antibody fragment, a heavy chain only antibody, a single-chain variable fragment (scFv), and an immunoglobulin single variable domain (ISV). In some embodiments one or more of the binding domains comprise a nanobody sequence. In some embodiments one or more of the binding domains comprise an scFv sequence.

In some embodiments the pair of cytokine adaptors comprise the amino acid sequences of SEQ ID NO:1 and SEQ ID NO:4, SEQ ID NO:16 and SEQ ID NO:17; SEQ ID NO:18 and SEQ ID NO:19; or a variant thereof, where the variant retains the binding functions of the proteins, but may comprise at least about 99% sequence identity to SEQ ID NO:1 and SEQ ID NO:4, SEQ ID NO:16 and SEQ ID NO:17; or SEQ ID NO:18 and SEQ ID NO:19, at least about 98% sequence identity, at least 97%, at least about 96%, at least about 95%, at least about 90%, at least about 80%, where variability may, for example and without limitation, be introduced into linker regions, framework regions, and the like.

In some embodiments a formulation is provided, comprising one or a pair of cytokine adapter proteins, and a pharmaceutically acceptable excipient. In some embodiments the cytokine adaptors are provided in a unit dose formulation. In some embodiments the pair of adaptors are co-formulated. In some embodiments the pair of adaptors are separately formulated. In some embodiments, the two cytokine adaptor components in a pair are linked together to form a single-component adaptor that can be administered in a single unit. The formulation can be provided as a kit, e.g. in combination with excipients, syringes, instructions for use, and the like.

In some embodiments, cytokine adaptors are administered in order to prevent, inhibit, or treat cancer in an individual in need thereof, comprising the steps of selecting an individual that has or is at risk of having cancer; and administering to the individual a composition having an effective amount of a cytokine adaptor and thereby preventing, inhibiting, or treating cancer in the individual. In some embodiments, the individual is a human. In some embodiments the cancer is a solid tumor. In some embodiments a solid tumor is analyzed for the concentration of the target binding protein of interest, e.g. a suppressive cytokine, prior to administration, where relatively high levels of the target binding protein is indicative that an individual is suitable for treatment. In an embodiment, the target binding domain binds to TGF-β, IL-10, IL-23, vascular endotheflal growth factor (VEGF), programmed death-ligand 1 (PDL1), human epidermal growth factor 2 (HER2), epidermal growth factor receptor (EGFR), fibroblast activation protein (FAP), tumor-associated calcium signal transducer 2 (Trop2), epithelial cell adhesion molecule (EPCAM), prostate-specific membrane antigen (PSMA), or arginase 2 (ARG2). In an embodiment, the target binding domain binds to TGFβ or IL-10. In an embodiment, the signal activator binding domain binds to IL-2Rβ or IL-2Rγ. Exemplary cytokine adapters for this purpose include, without limitation, the adapter pairs: CRG403 and CRG404; BS180 and BS181: BS182 and BS183; and the single component adapters: GA239, GA242, GA243, GA244.

In some embodiments, cytokine adaptors are administered in order to prevent, inhibit, or treat autoimmune disease in an individual in need thereof, comprising the steps of selecting an individual that has or is at risk of having an autoimmune disease, e.g. multiple sclerosis, insulin dependent diabetes mellitus, rheumatoid arthritis, psoriasis, systemic lupus erythematosus, etc. and administering to the individual a composition having an effective amount of a cytokine adaptor and thereby preventing, inhibiting, or treating the autoimmune disease in the individual. In some embodiments, the individual is a human. In some embodiments an autoimmune lesion, e.g. synovlal fluid for RA, pancreas for IDDM, CNS for MS, and the like is analyzed for the concentration of the target binding protein of Interest, e.g. a pro-inflammatory cytokine, prior to administration, where relatively high levels of the target binding protein is indicative that an individual is suitable for treatment. In an embodiment, the target binding domain binds to IL-23, IL-17A, IL-17B, IL-17C, IL-17C, IL-17D, IL-17E, IL-17F, IL-1β, IL-6, or TNF-α. In an embodiment, the target binding domain binds to IL-23. In an embodiment, the signal activator binding domain binds to IL-10Rα and IL-10Rβ. Exemplary cytokine adapters for this purpose include, without limitation, the adapter pair: KH319 and KH323.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Conceptual overview of cytokine adaptors. Cytokine adaptors are comprised of binding domains against an inhibitory cytokine (scFv, VHH, or soluble receptors) linked to binding domains against stimulatory cytokine receptors (scFv, VHH, or dominant negative cytokine). Cytokine adaptors convert an inhibitory signal into a stimulatory signal by blocking the inhibitory cytokine from binding to its receptor and instead compelling dimerization of the stimulatory receptor. (PDB: 2PJY, 2B5I).

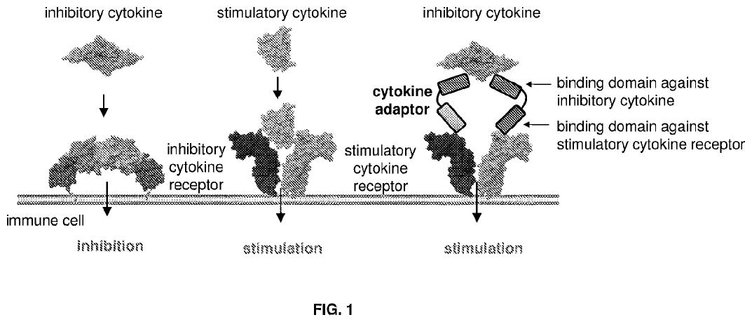

FIGS. 2A-2D. Cytokine adaptors CRG403 and CRG404 compel IL-2 receptor signaling in the presence of TGF-β. (A) Model of cytokine adaptors, comprised of TGF-β and scFv structure (PBD: 4KV5) overlayed with structures of IL-2RsNb6 bound to IL-2Rβ (PDB: 7S2S) and γcNb6 bound to γc (PDB:7S2R). (B) Schematic illustrating the design of TGF-β adaptors CRG403 and CRG404. (C) Dose-response curves for phospho-STAT5 in YT cell line stimulated for 20 minutes with human IL-2 or TGF-β with equimolar CRG403 and CRG404. (D) Dose-response curves for phospho-STAT5 in human T cell blasts stimulated for 20 minutes with human IL-2 or TGF-β with equimolar CRG403 and CRG404.

FIGS. 3A-3F. Single-component cytokine adaptor GA243 converts TGF-β stimulation to IL-2R signaling in human primary T cells. (A) Model illustrating the rationale for designing single-component cytokine adaptors containing a flexible linker, such that receptor dimerization is compelled only in the presence of TGF-β. (B) Schematic illustrating the design of single-component cytokine adaptors GA239, GA242, GA243, and GA244. GA239 contains N-terminal γ_{c}Nb6 and a 20aa linker, while GA242, GA243, and GA244 feature a reversed orientation and linker lengths of 10aa, 20aa, and 30aa respectively. In the single-component design of the cytokine adaptor, γ_{c}Nb6 and IL-2RβNb6 are distant and unstructured in the absence of TGF-β but are brought close to compel receptor dimerization and signaling in the presence of TGF-β. (C) Schematic illustrating downstream transcription factors activated by TGF-β signaling vs. IL-2R signaling. TGF-β receptor signaling leads to activation of pSMAD2 and pSMAD3 while IL-2R signaling leads to to activation of pSTAT5. (D) Mean fluorescence intensity of phospho-STAT5 and phospho-SMAD2iphospho-SMAD3 in human CD4 and CD8 T cell blasts treated with 10 nM TGF-β+/−GA239, GA242, GA243, or GA244. (E) Dose-response curves for phospho-STAT5 in human CD4 and CD8 T cell blasts stimulated for 20 minutes with human IL-2, GA243, TGF-β with equimolar GA243, or TGF-β with equimolar CRG403 and CRG404. (F) EC50 values for phospho-STAT5 were calculated from sigmoidal dose-response curves.

FIGS. 4A-4B. GA243 reverses T cell. Inhibition and promotes cytokine production in human primary T cells. (A-B) Human T cell blasts were pre-activated with anti-CD3 and anti-CD28 for 48 hours, rested overnight, then cultured for 6 days +/−500 ng/mL TGF-β+/− equimolar GA243, or IL-2 (1, 10, or 100 IU). On day 6, CD4 and CD8 T cell counts were quantified by flow cytometry (A), and CD8 T cells were treated with PMA, ionomycin, brefeldin A and monensin for 6 hours followed by intracellular cytokine staining for IFNγ and TNFα and analysis by flow cytometry (B).

FIGS. 5A-5D. Cytokine adaptors BS180-BS183 convert IL-10 stimulation to IL-2R signaling. (A) Structure of IL-10 and its receptors (PDB: 6X93) and model of cytokine adaptors, comprised of structures of IL-10 and scFv 9D7 (PDB: 1LK3) overlayed with IL-10 and IL-10Rα (PDB: 6X93), IL-2RβNb6 bound to IL-2Rβ (PDB: 7S2S) and γcNb6 bound to γc (PDB:7S2R). (B) Schematic illustrating the design of IL-10 adaptors BS180, BS181, BS182, and BS183. (C) Dose-response curves for phospho-STAT5 in YT cell line stimulated for 20 minutes with human IL-2 or IL-10 with equimolar BS181 and BS182, or BS180 and BS183. (D) Dose-response curves for phospho-STAT5 and phospho-STAT3 in human CD4 T cell blasts stimulated for 20 minutes with 10 nM IL-10 and increasing concentrations of equimolar BS180 and BS183.

FIGS. 6A-6C. IL-23 cytokine adaptors KH319 and KH323 convert IL-23 stimulation to IL-10 signaling. (A) Model of IL-23-IL-10 adaptors, comprised of IL-23p19 binder VHH 37D5 and IL-23p40 binder VHH 22E11 and IL-23p19 (PBD: 4GRW) overlayed with structures of IL-10 receptor complex (PDB: 6X93). (B) Schematic of IL-23-IL-10 cytokine adaptor design of molecules KH319 and KH323. (C) Dose-response curves for phospho-STAT3 in THP-1 cell line stimulated for 20 minutes with human IL-10 or IL-23 with equimolar IL-23 cytokine adaptors.

FIGS. 7A-7C. IL-23 adaptors suppress LIPS mediated inflammation. TNF-α (A), IL-6 (B), and IL-1β (C) quantified by ELISA in supernatants from human PBMCs stimulated with 24 hours with LPS and 10 nM IL-10, IL-23, KH319 and KH323.

### DETAILED DESCRIPTION OF THE EMBODIMENTS

In order for the present disclosure to be more readily understood, certain terms and phrases are defined below as well as throughout the specification. The definitions provided herein are non-limiting and should be read in view of what one of skill in the art would know at the time of invention.

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention. Is not entitled to antedate such publication by virtue of prior invention. Further, the dates of publication provided may be different from the actual publication dates which may need to be independently confirmed.

The term “identity,” as used herein in reference to polypeptide or DNA sequences, refers to the sequence identity between two molecules. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “polypeptide,” “protein” or “peptide” refer to any chain of amino acid residues, regardless of its length or post-translational modification (e.g., glycosylation or phosphorylation).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. Preferably, the variant polypeptide comprises at least one amino acid modification compared to the parent polypeptide, e.g. from about one to about ten amino acid modifications, and preferably from about one to about five amino acid modifications compared to the parent. A variant may be at least about 99% identical to the wild-type protein, at least about 98% Identical, at least about 97% identical, at least about 95% identical, at least about 90% identical.

By “parent polypeptide”, “parent protein”, “precursor polypeptide”, or “precursor protein” as used herein is meant an unmodified polypeptide that is subsequently modified to generate a variant polypeptide. A parent polypeptide may be a wild-type (or native) polypeptide. Parent polypeptide may refer to the polypeptide itself, compositions that comprise the parent polypeptide, or the amino acid sequence that encodes it.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, a “therapeutically effective amount” refers to that amount of the therapeutic agent, e.g. a pair of cytokine adaptors, a linked pair of cytokine adaptors, etc. sufficient to prevent, treat or manage a disease or disorder. A therapeutically effective amount may refer to the amount of therapeutic agent sufficient to delay or minimize the onset of disease, e.g., delay or minimize the spread of cancer or an inflammatory disease, or the amount effect to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “treating” is used to refer to treatment of a pre-existing condition. The treatment of ongoing disease, to stabilize or improve the clinical symptoms of the patient, is a particularly important benefit provided by the present invention. Evidence of therapeutic effect may be any diminution in the severity of disease, e.g. reduction of tumor size, decrease in residual disease, etc. The therapeutic effect can be measured in terms of clinical outcome or can be determined by immunological or biochemical tests. Patients for treatment may be mammals, e.g. primates, including humans, may be laboratory animals, e.g. rabbits, rats, mice, etc., particularly for evaluation of therapies, horses, dogs, cats, farm animals, etc.

As used herein, the terms “prevent”, “preventing” and “prevention” refer to the prevention of the recurrence or onset of one or more symptoms of a disorder in a subject as result of the administration of a prophylactic or therapeutic agent. In certain instances, prevention indicates inhibiting or delaying the onset of a disease or condition, in a patient identified as being at risk of developing the disease or condition.

As used herein, the terms “cancer” (or “cancerous”), “hyperproliferative,” and “neoplastic” to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Inflammatory Disease. Inflammation is a process whereby the immune system responds to infection or tissue damage. Inflammatory disease results from an activation of the immune system that causes illness, in the absence of infection or tissue damage, or at a response level that causes illness. Inflammatory disease includes autoimmune disease, which are any disease caused by immunity that becomes misdirected at healthy cells and/or tissues of the body. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

The immune system employs a highly complex mechanism designed to generate responses to protect mammals against a variety of foreign pathogens while at the same time preventing responses against self-antigens. In addition to deciding whether to respond (antigen specificity), the immune system must also choose appropriate effector functions to deal with each pathogen (effector specificity). Cells critical in mediating and regulating these effector functions are CD4+ T cells, which can be subtyped as TH1, TH2, TH17, etc.

Interleukin-23 (IL-23) is a pro-inflammatory cytokine produced by macrophages and dendritic cells in response to exogenous or endogenous signals, and drives the differentiation and activation of T helper 17 (Th17) cells with subsequent production of IL-17A, IL-17F, IL-6, IL-22, and tumor necrosis factor α (TNF-α). Its dysregulation has been shown to exacerbate chronic immune-mediated inflammation. Well-established experimental data support the concept that IL-23/IL-17 axis activation contributes to the development of inflammatory diseases including psoriasis, psoriatic arthritis; ankylosing spondylitis; inflammatory bowel disease; rheumatoid arthritis; Sjogren syndrome; and multiple sclerosis. Blockade of this pathogenic axis is a therapeutic target in several autoimmune disorders.

Th17 cells constitute a subset of effector T helper cells with distinct effector functions, and are identified as those T cells with a cytokine “signature” encompassing IL-17A, IL-17F, IL-22 and IL-26. Th17 cells can be potent inducers of tissue inflammation and have been associated with the pathogenesis of many experimental autoimmune diseases and human inflammatory conditions. Many TH-17 cells also express the signature TH1 cytokine, interferon γ.

Inflammatory diseases of interest include, without limitation Secondary Progressive Multiple Sclerosis (SPMS); Primary Progressive Multiple Sclerosis (PPMS); Neuromyelitis Optica (NMO); Psoriasis; Systemic Lupus Erythematosis (SLE); Ulcerative Colitis; Crohn's Disease; Ankylosing Spondylitis; Rheumatoid Arthritis (RA); Diabetes Mellitus type 1 (IDDM); Asthma; Chronic Obstructive Pulmonary Disorder (COPD); Chronic Hepatitis; Amyotrophic Lateral Sclerosis (ALS); Alzheimer's Disease (AD); Parkinson's Disease; Frontotemporal Lobar Degeneration (FTLD), atherosclerosis/cardiovascular disease, and obesity/metabolic syndrome.

In many autoimmune disorders, including SLE, psoriasis, RA and NMO, type I IFN contributes to the pathogenesis of the disease.

Inflammatory demyelinating disease. The term “inflammatory” response is the development of a humoral (antibody mediated) and/or a cellular (mediated by antigen-specific T cells or their secretion products) response. Inflammatory demyelinating diseases of the central nervous system are of interest and include, without limitation, multiple sclerosis (MS), neuromyelitis optica (NO), and experimental acquired encephalitis (EAE). Demyelinating inflammatory diseases of the peripheral nervous system include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

As used herein, the term “tumor-associated protein” refers to any protein that is associated with a tumor or has a higher concentration or expression in a tumor or within the local tumor environment. For instance, tumor proteins may be found in tissues outside of the tumor or may not be involved in non-tumor related processes such a cell expansion, differentiation or cell adhesion but may be at a higher than normal concentration in tumors relative to other tissues.

As used herein, the term “inflammation-associated protein” refers to a pro-inflammatory protein that may be associated with inflammatory lesions, or have a higher concentration or expression in such lesions.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

Interleukin 2 (IL-2) is a cytokine produced primarily by activated CD4+ T cells and plays a crucial role in producing a normal immune response. It is a type I α-helical cytokine that functions as a multi-lineage lymphocyte growth factor. On activated lymphocytes and T_{reg} cells, IL-2 signals through a high-affinity (10 pM) heterotrimeric receptor complex, consisting of the IL-2Rα (CD25), IL-2Rβ (CD122), and γc (CD132) chains. In resting lymphocytes it signals via the intermediate-affinity (1 nM) heterodimeric receptor complex, consisting of the IL-2Rβ and γc chains.

IL-2 promotes proliferation and expansion of activated T lymphocytes, and activates monocytes and natural killer cells. It was by virtue of these activities that IL-2 was tested and is used as an approved treatment of cancer (aldesleukin, Proleukin®). Human IL-2 is synthesized as a precursor polypeptide of 153 amino acids, from which 20 amino acids are removed to generate mature secreted IL-2. As used herein, “IL-2” refers to the native, or wild-type IL-2. Mature human IL-2 occurs as a 133 amino acid sequence (less the signal peptide, consisting of an additional 20 N-terminal amino acids), as described in Fujita, et. al, PNAS USA, 80, 7437-7441 (1983). The amino acid sequence of human IL-2 is found in Genbank under accession locator NP_000577.2.

IL-2 supports the survival and differentiation of T lymphocytes by initiating cell signaling pathways upon interaction with the IL-2 receptor (IL-2R). IL-2 is used clinically to treat a number of human diseases including cancer and autoimmunity and as an adjuvant to adoptive T cell therapies to promote the survival of transplanted T cells. However, IL-2 can also have opposing effects by activating off-target cell types.

IL-2 signal activation is achieved through the binding of the target signal activator binding domains to IL-2Rβ and IL-2Rγ following the binding of the target binding domain to TGF-β, leading to potent STAT5 phosphorylation. The studies of the present disclosure indicate that the presence of the pair of cytokine adaptors in the absence of TGF-β is not sufficient for STAT5 phosphorylation.

Transforming growth factor-beta (TGF-β) denotes a family of proteins, TGF-β1, TGF-β2, and TGF-β3, that are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication.

TGF-β exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGF-βR1) by the type II receptor (TGF-βR2).

Interleukin 10 (IL-10), also known as human cytokine synthesis inhibitory factor (CSIF), is an immunosuppressive cytokine, Genbank accession NP_000563.1. The protein is a homodimer; each of its subunits is 178-amino-acid long. IL-10 signals through a receptor complex consisting of two IL-10 receptor α (IL-10Rα) and two IL-10 receptor β (IL-10Rβ) proteins. IL-10 binding induces STAT3 signaling via the phosphorylation of the cytoplasmic tails of IL-10Rα and IL-10Rβ by JAK and Tyk2 respectively.

In humans, IL-10 is primarily produced by monocytes and, to a lesser extent, lymphocytes, particularly type-II T helper cells (Th2), mast cells, CD4+CD25+Foxp3+ regulatory T cells. IL-10 can be produced by monocytes upon PD-1 triggering in these cells. Expression of IL-10 is minimal in unstimulated tissues. It has multiple, pleiotropic, effects in immunoregulation and inflammation. It downregulates the expression of Th1 cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway.

Interleukin-23 is a heterodimeric cytokine composed of an IL12B (IL-12p40) subunit shared with IL12 and the IL23A (IL-23p19) subunit, see Genbank refseq NP_057668. The IL-23 receptor is composed of IL-12Rβ1 and IL-23R. The protein comprises a signal peptide, an N-terminal fibronectin III-like domain and an intracellular domain with 3 potential tyrosine phosphorylation domains, and is 629 amino acids in length. IL-23 is mainly secreted by activated dendritic cells, macrophages or monocytes. IL-23 is associated with autoimmune and cancerous diseases.

The p19 subunit of IL-23 binds IL-23R while p40 subunit binds IL-12Rβ1 which activates receptor-associated JAK2 and TYK2 leading to the phosphorylation of STAT3. STATs dimerise and activate transcription of target genes in nucleus. STAT3 is responsble for key Th17 development attributes like RORγt expression or transcription of Th17 cytokines.

VEGF is a dimeric, disulfide-linked 46-kDa glycoprotein related to Platelet-Derived Growth Factor (“PDGF”). It is produced by normal and cancerous cells; is an endothelial cell-selective mitogen; shows angiogenic activity in in vivo test systems (e.g., rabbit cornea); is chemotactic for endothelial cells and monocytes; and induces plasminogen activators in endothelial cells, which are involved in the proteolytic degradation of the extracellular matrix during the formation of capillaries. A number of isoforms of VEGF are known, which while they show comparable biological activity, differ in the type of cells that secrete them and in their heparin-binding capacity. In addition, there are other members of the VEGF family, such as Placenta Growth Factor (“PGF”) and VEGF-C.

The cellular receptors of VEGFs (VEGFRs) are transmembranous receptor tyrosine kinases. They are characterized by an extracellular domain with seven immunoglobulin-like domains and an intracellular tyrosine kinase domain. Various types of VEGF receptor have been characterized, Including VEGFR-1 (also known as flt-1), VEGFR-2 (also known as KDR), and VEGFR-3.

Interleukin 17 refers to a family of pro-inflammatory cystine knot cytokines, usually produced by T helper cells. The IL-17 family in humans comprises IL17A, IL17B, IL17C, IL17D, IL17E and IL17F. IL-17E is also known as IL-25. The most notable role of IL-17 is its involvement in inducing and mediating proinflammatory responses.

IL-17(A) is a 155-amino acid protein that is a disulfide-linked, homodimeric, secreted glycoprotein with a molecular mass of 35 kDa. Each subunit of the homodimer is approximately 15-20 KDa. The structure of IL-17 consists of a signal peptide of 23 amino acids (aa) followed by a 123-aa chain region characteristic of the IL-17 family. An N-linked glycosylation site on the protein was first identified after purification of the protein revealed two bands, one at 15 KDa and another at 20 KDa. Comparison of different members of the IL-17 family revealed four conserved cysteines that form two disulfide bonds. IL-17 is unique in that it bears no resemblance to other known interleukins. Furthermore, IL-17 bears no resemblance to any other known proteins or structural domains.

The IL-17 receptor family consists of five, broadly distributed receptors (IL-17RA, B, C, D and E) that present with individual ligand specificities. Within this family of receptors, IL-17RA is the best-described. IL-17RA binds both IL-17A and IL-17F and is expressed in multiple tissues: vascular endothelial cells, peripheral T cells, B cell lineages, fibroblast, lung, myelomonocytic cells, and marrow stromal cells. Signal transduction for both IL-17A and IL-17F requires the presence of a heterodimeric complex consisting of both IL-17RA and IL-17RC. IL-17RB, binds both IL-17B and IL-17E.

After binding to its receptor, IL-17 activates signaling cascades that can lead to the induction of chemokines, and recruitment of immune cells. Activation of IL-17 signaling can be associated with allergic responses, and autoimmune disorders, including rheumatoid arthritis, asthma, lupus, allograft rejection, anti-tumour immunity, psoriasis, multiple sclerosis, and intracerebral hemorrhage.

The IL-23/IL-17 pathway plays a major role in psoriasis. Analysis of biopsies taken from lesions of psoriasis patients show an enrichment of cytotoxic T cells and neutrophils containing IL-17, indicating that an infiltration of pro-inflammatory immune cells and IL-17 cytokines are associated with the development of psoriasis. Removing IL-23 or IL-17 decreases the progression of psoriasis. IL-17 promotes psoriasis by contributing to the inflammatory response that damages and overturns the keratinocyte cells of the epidermal layer. IL-17, particularly IL-17A is a therapeutic target for psoriasis

IL-17F has a pro-inflammatory role in asthma, and its expression level is correlated with disease severity. A coding region variant (H161R) of the IL-17F gene is inversely associated with asthma and encodes an antagonist for the wild-type IL-17F. IL-17F induces several cytokines, chemokines and adhesion molecules in bronchial epithelial cells, vein endothelial cells, fibroblasts and eosinophils. IL-17F has important therapeutic implications in asthma.

Because of its involvement in immune regulatory functions, IL-17 inhibitors are being investigated as possible treatments for autoimmune diseases such as rheumatoid arthritis, psoriasis and inflammatory bowel disease. For example, secukinumab is approved for treatment of moderate to severe plaque psoriasis; and the anti-IL-23 antibody ustekinumab is used to treat psoriasis by indirectly reducing IL-17.

The refseq for human IL-17 proteins may be accessed at Genbank as follows: IL-17A NP_002181; IL-17B, NP_055258; IL-17C, NP_037410; IL-17D, NP_612141; IL-17E, NP_073626; IL-17F, NP_443104.

Interleukin-10 receptor (IL-10R) is a type II cytokine receptor. The receptor is tetrameric, composed of 2α and 2β subunits. The a subunit (encoded in the IL10ra gene) is expressed on immune cells, e.g. T. B, NK, mast, and dendritic cells. The β subunit (encoded in the IL10rb gene) is expressed ubiquitously. The a subunit is exclusive to interleukin-10, however the β subunit is shared with other type II cytokine receptors, e.g. IL-22R, IL-26R and INFAR.

Signaling through the IL-10 receptor (IL-10R) via induction of STAT3 generates a largely immunosuppressive response, with signaling leading to suppression of macrophage and DC function, and in turn, reduction of pathogenic T-cell responses. IL-10R signaling has a key role to play in the Treg/Th17 axis: IL-10 signaling suppresses the Th17 axis by inhibiting the generation and proliferation of Th17 cells, and promoting Tregs.

As used herein the term “specifically binds” refers to the degree of selectivity or affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, at least ten times greater, at least 20-times greater, or at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the affinity of the antibody for the second molecule of the binding pair is higher than about 10⁹ liters/mole, alternatively higher than about 10¹⁰ liters/mole, higher than about 10¹¹ liters/mole, higher than about 10¹² liters/mole as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays, conducted at room temperature.

Binding domains of interest include, for example, designed binding proteins, ligands, antibodies and related binding proteins, and the like. In some embodiments a binding domain is an antibody, antibody fragment, or variant thereof. The term “antibody” herein is used in the broadest sense and specifically covers monoclonal antibodies, polyclonal antibodies, monomers, dimers, multimers, heavy chain only antibodies, three chain antibodies, single chain Fv, single domain antibodies, NANOBODIES® etc., and also include antibody fragments with or without pegylation, so long as they exhibit the desired biological activity (Miller et al (2003) Jour. of Immunology 170:4854-4861).

The term antibody may reference a full-length heavy chain, a full length light chain, an intact immunoglobulin molecule including a functional Fc sequence; or an immunologically active portion of any of these polypeptides, i.e., a polypeptide that comprises an antigen binding site that immunospecifically binds an antigen of a target of interest or part thereof.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodles or domain antibodies comprising single Ig domains from human or non-human species or other specific single-domain binding modules including non-antibody binding proteins such as, but not limited to, adnectins and anticalins; and multispecific or multivalent structures formed from antibody fragments.

The term “NANOBODY®” as used herein refers to a single domain antibody consisting of a single monomeric variable domain (also referred to as a variable heavy homodimer [V_{HH}] domain or immunoglobulin single variable domains or ISVs). The single domain antibodies are naturally produced by animals belonging to the camelid family. Nanobodies are smaller than human antibodies, where ISV are generally 12-15 kDa, human antibodies are generally 150-160 kDa, Fab fragments are ˜50 kDa and single-chain variable fragments are ˜25 kDa. NANOBODIES® provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like.

Immunoglobulin sequences, such as antibodies and antigen binding fragments derived therefrom (e.g. ISVs,) are used to specifically target the respective antigens disclosed herein. The generation of immunoglobulin single variable domains such as e.g., V_{HH}s or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

Alternatively, similar immunoglobulin single variable domains can be generated and selected by the immunization of an experimental animal such as a llama, construction of phage libraries from immune tissue, and

Unless indicated otherwise, the term “immunoglobulin single variable domain” or “ISV” is used as a general term to include but not limited to antigen-binding domains or fragments such as V_{HH} domains or V_{H} or V_{L} domains, respectively. The terms antigen-binding molecules or antigen-binding protein are used interchangeably and include also the term NANOBODIES®. The immunoglobulin single variable domains can be light chain variable domain sequences [e.g., a V_{L}-sequence), or heavy chain variable domain sequences (e.g., a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES®, including but not limited to V_{H} sequences. An amino acid sequence such as e.g. an immunoglobulin single variable domain or polypeptide is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195.

The Invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. An immunoglobulin variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively.

Such immunoglobulin single variable domains may be derived in any suitable manner and from any suitable source, and may for example be naturally occurring V_{HH} sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VH Hs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

Linker. The target binding domain and the target signal activator binding domain may be separated by a linker, e.g. a polypeptide linker, or a non-peptidic linker, etc. The amino acid linkers that join domains can play an important role in the structure and function of multi-domain proteins. There are numerous examples of proteins whose catalytic activity requires proper linker composition. In general, altering the length of linkers connecting domains has been shown to affect protein stability, folding rates and domain-domain orientation (see George and Hering (2003) Prot. Eng. 15:871-879). The length of the linker in the cytokine adaptor, and therefore the spacing between the binding domains, can be used to modulate the signal strength of the cytokine adaptor, and can be selected depending on the desired use of the cytokine adaptor. The enforced distance between binding domains of a cytokine adaptor can vary, but in certain embodiments may be less than about 100 angstroms, less than about 90 angstroms, less than about 80 angstroms, less than about 70 angstroms, less than about 60 angstroms, less than about 50 angstroms.

In some embodiments the linker is a rigid linker. In other embodiments the linker is a flexible linker. In some embodiments, the linker moiety is a peptide linker. In some embodiments, the peptide linker comprises 2 to 100 amino acids. In some embodiments, the peptide linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 but no greater than 100 amino acids. In some embodiments, the peptide linker is between 5 to 75, 5 to 50, 5 to 25, 5 to 20, 5 to 15, 5 to 10 or 5 to 9 amino acids in length. Exemplary linkers include linear peptides having at least two amino acid residues such as Gly-Gly. Gly-Ala-Gly, Gly-Pro-Ala, (SEQ ID NO:8) Gly-Gly-Gly-Gly-Ser. Suitable linear peptides include poly glycine, polyserine, polyproline, polyalanine and oligopeptides consisting of alanyl and/or serinyl and/or prolinyl and/or glycyl amino acid residues. In some embodiments, the peptide linker comprises the amino acid sequence selected from the group consisting of Gly₉, Glu₉, Ser₉, (SEQ ID NO:20) Gly₅-Cys-Pro₂-Cys, (Gly₄-Ser)₃, (SEQ ID NO:21) Ser-Cys-Val-Pro-Leu-Met-Arg-Cys-Gly-Gly-Cys-Cys-Asn, (SEQ ID NO:22) Pro-Ser-Cys-Val-Pro-Leu-Met-Arg-Cys-Gly-Gly-Cys-Cys-Asn, (SEQ ID NO:23) Gly-Asp-Leu-Ile-Tyr-Arg-Asn-Gln-Lys, and (SEQ ID NO:24) Gly₉-Pro-Ser-Cys-Val-Pro-Leu-Met-Arg-Cys-Gly-Gly-Cys-Cys-Asn. In one embodiment a linker comprises the amino acid sequence (SEQ ID NO:25) GSTSGSGKSSEGKG, or (SEQ ID NO:8) (GGGGS)n, where n is 1, 2, 3, 4, 5, etc.; however many such linkers are known and used in the art and may serve this purpose.

Cytokine adaptors can be provided in single-chain form, which means that the binding domains are linked by peptide bonds through a linker peptide. In other embodiments, the binding domains are individual peptides and can be joined through a non-peptidic linker. Pairs of cytokine adaptors can also be joined, e.g. through a linker peptide or non-peptidic linker.

Chemical groups that find use in linking binding domains include carbamate; amide (amine plus carboxylic acid); ester (alcohol plus carboxylic acid), thioether (haloalane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The linkage between binding domains may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)n where n is from 1 to about 12; (CH₂CH₂NH)n, where n is from 1 to about 12; [(CH₂)n(C═O)NH(CH₂)ₘ]_{z}, where n and m are from 1 to about 6, and z is from 1 to about 10; [(CH₂)nOPO; (CH₂)ₘ]_{z} where n and m are from 1 to about 6, and z is from 1 to about 10. Such linkers may include polyethylene glycol, which may be linear or branched.

The binding domains may be joined through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde. NHS-PEG-MAL; succinimidyl 4-[N-maleimidomethyl]cyclohexane-1-carboxylate; 3-(2-pyridyldithio)propionic acid N-hydroxysuccinimide ester (SPDP); N, N′-(1,3-phenylene) bismaleimide; N, N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenyisulfone (which forms irreversible cross-linkages with amino and phenolic groups); dimethyl adipimidate (which is specific for amino groups); phenol-1,4-disulfonylchloride (which reacts principally with amino groups); hexamethylenedilsocyanate or dilsothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

A cytokine adaptor may comprise a sequence:

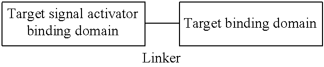

where the binding domains and linker are as described above. In some embodiments the target binding domain binds to human TGFβ. Such binding domains are known in the art, or may be generated de novo. For example, ScFvs that bind to TGF-β, such as GC1008, have been described in US 2016/0017026 A1, incorporated here in its entirety.

In some embodiments the target signal activator binding domain specifically binds to human IL-2 receptors, e.g. IL-2Rβ and IL-2Rγ. Such binding domains are known in the art or may be generated de novo, for example see Silva et al. (2019) Nature 565(7738): 186-191. In other embodiments the target signal activator binding domain specifically binds to human IL-10 receptors, e.g. IL-10a and IL-10p.

In some embodiments, the target binding domain binds a tumor-associated protein of interest. Tumor-associated proteins that find use in the present disclosure include, without limitation, IL-10, IL-23, vascular endothelial growth factor (VEGF), programmed death-ligand 1 (PDL1), human epidermal growth factor 2 (HER2), epidermal growth factor receptor (EGFR), fibroblast activation protein (FAP), tumor-associated calcium signal transducer 2 (Trop2), epithelial cell adhesion molecule (EPCAM), prostate-specific membrane antigen (PSMA), arginase 2 (ARG2).

In some embodiments the target binding domain binds a proinflammatory cytokine, e.g. IL23, IL-17A, IL-17B, IL-17C, IL-17C, IL-17D, IL-17E, IL-17F, IL-10, IL-6, TNF-α, etc.

For a target binding domain that binds to an IL-10 protein, exemplary antigen binding sequences that find use in the present disclosure can be found in US 2014/0112919 A1, incorporated herein in its entirety.

For a target binding domain that binds to an IL-23 protein, exemplary antigen binding sequences that find use in the present disclosure can be found in Desmyter et al. Front Immunol. 2017; 8: 884, or Roberts et al. Sci Rep. 2021 Sep. 30; 11(1):19422. doi: 10.1038/s41598-021-97236-0, incorporated herein in their entirety.

For a target binding domain that binds to a VEGF protein, exemplary antigen binding sequences that find use in the present disclosure can be found in Sadeghi et al. Drug Test Anal. 2020 January; 12(1):92-100. doi: 10.1002/dta.2693, Kazemi-Lomedasht et al. Iran J Basic Med Sci. 2018 March; 21(3): 260-266, or Karami et al. J Enzyme Inhib Med Chem. 2020; 35(1): 1233-1239, incorporated herein in their entirety.

For a target binding domain that binds to a PD-L1 protein, exemplary antigen binding sequences that find use in the present disclosure can be found in US 2021/0284737 A1 or US 2021/0269528 A1, incorporated herein in their entirety.

For a target binding domain that binds to a HER2 protein, exemplary antigen binding sequences that find use in the present disclosure can be found in US 2021/0299269, US 2021/0290676, US 2021/0137977 A1, US 2021/01016620 A1, or US 2021/0299172 A1, incorporated herein in their entirety.

For a target binding domain that binds to an EGFR protein, exemplary antigen binding sequences that find use in the present disclose can be found in US 2021/0290676, US 2021/0269547 A1, US 2021/0155702 A1, Xia et al. Clin Transl Immunology. 2020 May 3;9(5):e01135, Li et al. Cell Death Dis. 2018 February; 9(2): 177, or Liu et al. Clinical Trial Cytotherapy 2020 October; 22(10):573-580 incorporated herein in their entirety.

For a target binding domain that binds to a FAP protein exemplary, antigen binding sequences that find use in the present disclosure can be found in US 2021/0252122 A1, Kakarla et al. Mol Ther. 2013 August; 21(8):1611-20, Wang et al. Cancer Immunol Res. 2015 July; 3(7): 815-826, Petrausch et al. BMC Cancer. 2012; 12: 615, or Tran et al. J Exp Med. 2013 Jun. 3; 210(6):1125-35, incorporated herein in their entirety.

For a target binding domain that binds to a Trop2 protein, exemplary antigen binding sequences that find use in the present disclosure can be found in US 2021/0290676, Zhao et al. Am J Cancer Res. 2019; 9(8): 1846-1856, Bedoya et al. Cytotherapy 2019 May; 21(5): S11-12, or Sayama et al. Mol Med Rep. 2021 February; 23(2):92, incorporated herein in their entirety.

For a target binding domain that binds to an EPCAM protein, exemplary antigen binding sequences that find use in the present disclosure can be found in US 2021/0290676, US 2021/0284728 A1, US 2021/0269547 A1, Qin et al. Oncoimmunology. 2020 Aug. 15; 9(1):1806009, or Deng et al. BMC Immunol. 2015 Jan. 31; 16(1):1, incorporated herein in their entirety.

For a target binding domain that binds to a PSMA protein, exemplary antigen binding sequences that find use in the present disclosure can be found in US 2021/0290676, US 2021/0284728 A1, US 2021/0269547 A1, US 2021/0252122 A1, US 2021/0137977 A1, or US 2021/0113615 A1, incorporated herein in their entirety.

For a target binding domain that binds to an ARG2 protein, exemplary antigen binding sequences that find use in the present disclosure can be found in Chan et al. Proc Natl Acad Sci USA. 2020 Jul. 21; 117(29): 16949-16960, incorporated herein in their entirety.

In some embodiments, a cytokine adaptor target binding domain comprises an scFv GC1008 sequence, which binds to human TGFβ. The target binding domain can be fused to a target signal activator binding domain comprising a nanobody that specifically binds to IL-2Rβ. Such a cytokine adaptor comprises the sequence (SEQ ID NO:1) QVOLQESGGGSVQAGGSLRLSCAASSYTISSVCMGWFRQAPGKEREGVAGIAPDGSTGYGD SVKGRFTISKDNAKNTLYLQMNSLKPEDTAMYYCAAASPGRCFLPRTALEPALYYNWGQGTO VTVSSQVQLVQSGAEVKKPGSSVKVSCKASGYTFSSNVISWVRQAPGQGLEWMGGVIPIVDI ANYAQRFKGRVTITADESTSTTYMELSSLRSEDTAVYYCASTLGLVLDAMDYWGOGTLVTVSS GGGGSGGGGSGGGGSALETVLTQSPGTLSLSPGERATLSCRASQSLGSSYLAWYQQKPGQ APRLLIYGASSRAPGIPDRFSGSGSGTDFTLTISRLEPEDFAVYYCQQYADSPITFGQGTRLEIK RHHHHHH, where a linker is optionally inserted between residues 128 and 129, and optionally lacking the terminal poly-histidine tag.

In some embodiments, a target binding domain comprising scFv GC1008 comprises the amino acid sequence: QVQLVQSGAEVKKPGSSVKVSCKASGYTFSSNVISWVRQAPGQGLEWMGGVIPIVDIANYAQ RFKGRVTITADESTSTTYMELSSLRSEDTAVYYCASTLGLVLDAMDYWGQGTLVTVSSGGGG SGGGGSGGGGSALETVLTQSPGTLSLSPGERATLSCRASQSLGSSYLAWYQQKPGQAPRLLI YGASSRAPGIPDRFSGSGSGTDFTLTISRLEPEDFAVYYCQQYADSPITFGQGTRLEIKR (SEQ ID NO: 2). ScFvs that bind to TGF-β, such as GC1008, have been described in US 2016/0017026 A1, incorporated here in its entirety.

In some embodiments, the target signal activator binding domain comprising the nanobody that specifically binds to IL-2Rβ comprises the amino acid sequence: QVQLQESGGGSVQAGGSLRLSCAASSYTISSVCMGWFRQAPGKEREGVAGIAPDGSTGYGD SVKGRFTISKDNAKNTLYLQMNSLKPEDTAMYYCAAASPGRCFLPRTALEPALYYNWGQGTQ VTVSS (SEQ ID NO: 3).

In some embodiments, a cytokine adaptor comprises scFv GC1008 fused to a target signal activator domain comprising a nanobody that specifically binds to IL-2Rγ having the sequence of (SEQ ID NO: 4) QVQLQESGGGSVQAGGSLRLSCAASGYTYRDYYMGWFRQAPGREREGVASIYTRGSREGS TRYSSSVEGRFTITLDTAKNTLYLQMNSLKPEDTAMYYCAADDRTWLPRVQLGGPRENEYNY WGQGTQVTVSSQVQLVQSGAEVKKPGSSVKVSCKASGYTFSSNVISWVRQAPGQGLEWMG GVIPIVDIANYAQRFKGRVTITADESTSTTYMELSSLRSEDTAVYYCASTLGLVLDAMDYWGQG TLVTVSSGGGGSGGGGSGGGGSALETVLTQSPGTLSLSPGERATLSCRASQSLGSSYLAWY QQKPGQAPRLLIYGASSRAPGIPDRFSGSGSGTDFTLTISRLEPEDFAVYYCQQYADSPITFGQ GTRLEIKRHHHHHH, and optionally lacking the terminal poly-histidine tag.

In some embodiments, the target signal activator binding domain comprising the nanobody which specifically binds to IL-2Rγ comprises the amino acid sequence: QVQLQESGGGSVQAGGSLRLSCAASGYTYRDYYMGWFRQAPGREREGVASIYTRGSREGS TRYSSSVEGRFTITLDTAKNTLYLQMNSLKPEDTAMYYCAADDRTWLPRVQLGGPRENEYNY WGQGTQVTVSS.

In some embodiments, the cytokine adaptors can be conjugated to additional molecules to provide desired pharmacological properties such as extended half-life. In some embodiments a cytokine adaptor is conjugated to a polyethylene glycol molecules or “PEGylated.” The molecular weight of the PEG conjugated to the cytokine adaptor include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the cytokine adaptor polypeptide or via a linker molecule. The processes and chemical reactions necessary to achieve PEGylation of biological compounds is well known in the art.

Cytokine adaptors can be acetylated at the N-terminus, using methods known in the art, e.g. by enzymatic reaction with N-terminal acetyltransferase and, for example, acetyl CoA. Cytokine adaptors can be acetylated at one or more lysine residues, e.g. by enzymatic reaction with a lysine acetyltransferase. See, for example Choudhary et al. (2009). Science. 325 (5942): 834-840.

In other embodiments, a cytokine adaptor polypeptide can comprise polypeptide that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In some embodiments, the chimeric polypeptide further comprises a C-terminal c-myc epitope tag.

As described above, the cytokine adaptor proteins of the invention may exist as a part of a chimeric polypeptide. In addition to, or in place of, the heterologous polypeptides described above, a nucleic acid molecule of the invention can contain sequences encoding a “marker” or “reporter.” Examples of marker or reporter genes include a C-terminal HIS tag, β-lactamase, chloramphenicol acetyltransferase (CAT), adenosine deaminase (ADA), aminoglycoside phosphotransferase (neo1, G418r), dihydrofolate reductase (DHFR), hygromycin-β-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

Cytokine adaptors may also include conservative modifications and substitutions at other positions of the cytokine (e.g. positions other than those involved in the cytokine adaptor engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gin, asn, ser, thr; Group II: cys, ser, tyr, thr; Group III; val, ile, leu, met, ala, phe; Group IV: lys, arg, his; Group V: phe, tyr, trp, his; and Group VI: asp, glu. In each instance, the introduction of additional modifications may be evaluated to minimize any increase in antigenicity of the modified polypeptide in the organism to which the modified polypeptide is to be administered.

A cytokine adaptor protein may be produced by recombinant methods. The cytokine adaptor may be introduced on an expression vector into the cell to be engineered. DNA encoding a cytokine adaptor protein may be obtained from various sources as designed during the engineering process.

Amino acid sequence variants are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

To achieve expression of the recombinant protein, a nucleic acid encoding a cytokine adaptor protein is inserted into a replicable vector for expression. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors for expression of the cytokine adaptor in a cell may be viral vectors or non-viral vectors. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated iposomes (Marin et al., U.S. Pat. No. 5,213,804, issued May 25, 1993; Woodie, et al., U.S. Pat. No. 5,013,556, issued May 7, 1991); cationic liposomes (Epand et al., U.S. Pat. No. 5,283,185, issued Feb. 1, 1994: Jessee, J. A., U.S. Pat. No. 5,578,475, issued Nov. 26. 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. When a viral vector system is to be employed for expression of the cytokine adaptor, retroviral or lentiviral expression vectors are preferred. In particular, the viral vector is a gamma retrovirus (.(Pule, et al. (2008) Nature Medicine 14(11):1264-1270), self-inactivating lentiviral vectors (June, et al. (2009) Nat Rev Immunol 9(10):704-716) and retroviral vectors as described in Naldini, et al. (1996) Science 272: 263-267; Naldini, et al. (1996) Proc. Natl. Acad. Sci. USA Vol. 93, pp. 11382-11388; Dull, et al. (1998) J. Virology 72(11):8463-8471; Milone, et al. (2009) 17(8):1453-1464; Kingsman, et al. U.S. Pat. No. 6,096,538 issued Aug. 1, 2000 and Kingsman, et al. U.S. Pat. No. 6,924,123 issued Aug. 2, 2005.

A cytokine adaptor protein may be produced recombinantly as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cel. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to a cytokine adaptor protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryote, yeast, or higher eukaryote cells described above. Examples of useful mammalian host cell lines are mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells can be transfected with the above-described expression vectors for cytokine adaptor expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Recombinantly produced cytokine adaptor polypeptides can be recovered from the culture medium as a secreted polypeptide, although it can also be recovered from host cell lysates. A protease inhibitor, such as phenyl methyl sulfonyl fluoride (PMSF) also may be useful to inhibit proteolytic degradation during purification, and antibiotics may be included to prevent the growth of adventitious contaminants. Various purification steps are known in the art and find use, e.g. affinity chromatography, such as Nickel-NTA to purify proteins with a His-tag followed by size exclusion chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column. Also of interest is cation exchange chromatography.

The cytokine adaptor composition may be concentrated, filtered, dialyzed, etc., using methods known in the art. For therapeutic applications, the cytokine adaptors can be administered to a mammal comprising the appropriate cytokine adaptor pair. Administration may be intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The cytokine adaptors also are suitably administered by intratumoral, peritumoral, intralesional, or perilesional routes or to the lymph, to exert local as well as systemic therapeutic effects.

Such dosage forms encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methykcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The polypeptide will typically be formulated in such vehicles at a concentration of about 0.1 μg/ml to 100 μg/ml.

In the event the cytokine adaptor polypeptides of the disclosure are “substantially pure,” they can be at least about 60% by weight (dry weight) the polypeptide of interest, for example, a polypeptide containing the cytokine adapter sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition is the cytokine adaptor. The label on, or associated with, the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

The preferred formulation depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldlmethylbenzyl ammonium chloride; hexamethonium chloride; benzalkonlum chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### Pharmaceutical Compositions

For therapeutic applications, the cytokine adaptors are administered to a mammal, preferably a human, in a physiologically acceptable dosage form, Including those that may be administered to a human intravenously as a bolus or by continuous infusion over a period of time. Alternative routes of administration include topical, intramuscular, intraperitoneal, intra-cerobrospinal, subcutaneous, intra-articular, intrasynovial, intrathecal, oral, topical, or inhalation routes. The cytokine adaptors also are suitably administered by intratumoral, peritumoral, intralesional, or perilesional routes or to the lymph, to exert local as well as systemic therapeutic effects.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, Ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugarcoated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will be different from patient to patient. A formulation may be provided, for example, in a unit dose. A competent clinician will be able to determine an effective amount of a therapeutic agent to administer to a patient. Dosage of the surrogate will depend on the treatment, route of administration, the nature of the therapeutics, sensitivity of the disease to the therapeutics, etc. Utilizing LD₅₀ animal data, and other information available, a clinician can determine the maximum safe dose for an individual, depending on the route of administration. Compositions which are rapidly cleared from the body may be administered at higher doses, or in repeated doses, in order to maintain a therapeutic concentration. Utilizing ordinary skill, the competent clinician will be able to optimize the dosage of a particular therapeutic or imaging composition in the course of routine clinical trials. Typically the dosage will be 0.001 to 100 milligrams of agent per kilogram subject body weight.

The compositions can be administered to the subject in a series of more than one administration. For therapeutic compositions, regular periodic administration (e.g., every 2-3 days) will sometimes be required, or may be desirable to reduce toxicity. For therapeutic compositions which will be utilized in repeated-dose regimens, moieties which do not provoke immune responses are preferred.

In another embodiment of the invention, an article of manufacture containing materials useful for the treatment of the conditions described herein is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition is the cytokine adaptor. The label on, or associated with, the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

For example, in some embodiments, term “therapeutically effective amount”, refers to an amount which, when administered to an individual in need thereof in the context of inventive therapy, will block, stabilize, attenuate, or reverse a disease process occurring in said individual.

Formulations to be used for in vivo administration are typically sterile. Sterilization of the compositions of the present invention may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Also provided are kits for use in the methods. The subject kits include an expression vector encoding a cytokine adaptor, or a cell comprising the expression vector. In some embodiments, the components are provided in a dosage form (e.g., a therapeutically effective dosage form), in liquid or solid form in any convenient packaging (e.g., stick pack, dose pack, etc.). Reagents for the selection or in vitro derivation of cells may also be provided, e.g. growth factors, differentiation agents, tissue culture reagents; and the like.

In addition to the above components, the subject kits may further include (In certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Methods of Use

The dosage of the therapeutic formulation, e.g., pharmaceutical composition, will vary widely, depending upon the nature of the condition, the frequency of administration, the manner of administration, the clearance of the agent from the host, and the like. In particular embodiments, the initial dose can be larger, followed by smaller maintenance doses. In certain embodiments, the dose can be administered as infrequently as weekly or biweekly, or more often fractionated into smaller doses and administered daily, semi-weekly, or otherwise as needed to maintain an effective dosage level.

In some embodiments of the invention, administration of the composition or formulation comprising the cytokine adaptors is performed by local administration. Local administration, as used herein, may refer to topical administration, but also refers to injection or other introduction into the body at a site of treatment. Examples of such administration include intratumoral injection, intramuscular injection, subcutaneous injection, intraperitoneal injection, and the like. In other embodiments, the composition or formulation comprising the cytokine adaptors is administered systemically, e.g., orally or intravenously. In one embodiment, the composition of formulation comprising the cytokine adaptors is administered by infusion, e.g., continuous infusion over a period of time, e.g., 10 min, 20 min, 3 min, one hour, two hours, three hours, four hours, or greater.

In some embodiments of the invention, the compositions or formulations are administered on a short term basis, for example a single administration, or a series of administrations performed over, e.g. 1, 2, 3 or more days, up to 1 or 2 weeks, in order to obtain a rapid, significant increase in activity. The size of the dose administered must be determined by a physician and will depend on a number of factors, such as the nature and gravity of the disease, the age and state of health of the patient and the patient's tolerance to the drug itself.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an adapter pair is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the adapter pair in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent. The dosage may also be varied for localized administration, e.g. intranasal, inhalation, etc., or for systemic administration, e.g. i.m., i.p., i.v., and the like.

In certain methods of the present invention, an effective amount of a composition comprising a pair of cytokine adaptors is provided to cells, e.g. by contacting the cell with an effective amount of that composition to achieve a desired effect, e.g. to initiate a pro-inflammatory response, etc. In particular embodiments, the contacting occurs in vitro, ex vivo or in vivo. In particular embodiments, the cells are derived from or present within a subject in need of a pro-inflammatory response.

In some methods of the invention, an effective amount of the subject composition is provided to induce a pro-inflammatory response in a cell, tissue or an individual. Biochemically speaking, an effective amount or effective dose of cytokine adaptors is an amount to induce a pro-inflammatory response in a cell, tissue or individual by at least 30%, at least 40%, at least 50%, at least 60%, at least 70%, at least 80%, at least 90%, at least 95%, or by 100% relative to the signaling in the absence of the cytokine adaptors. The amount of modulation of a cell's activity can be determined by a number of ways known to one of ordinary skill in the art of immunology.

In a clinical sense, an effective dose of a cytokine adaptor composition is the dose that, when administered to a subject for a suitable period of time, e.g., at least about one week, and may be about two weeks, or more, up to a period of about 4 weeks, 8 weeks, or longer, will evidence an alteration in the symptoms associated with a lack of a pro-inflammatory response. In some embodiments, an effective dose may not only slow or halt the progression of the disease condition but may also induce the reversal of the condition. It will be understood by those of skill in the art that an initial dose may be administered for such periods of time, followed by maintenance doses, which, in some cases, will be at a reduced dosage.

The calculation of the effective amount or effective dose of cytokine adaptor composition to be administered is within the skill of one of ordinary skill in the art, and will be routine to those persons skilled in the art. Needless to say, the final amount to be administered will be dependent upon the route of administration and upon the nature of the disorder or condition that is to be treated.

Cells in vivo may be contacted with the subject cytokine adaptor compositions by any of a number of well-known methods in the art for the administration of peptides, small molecules, or nucleic acids to a subject. The cytokine adaptor composition can be incorporated into a variety of formulations or pharmaceutical compositions, which in some embodiments will be formulated in the absence of detergents, liposomes, etc.

Conditions of interest for treatment with the compositions of the invention include, without limitation, a number of different types of cancers. Examples of cancers that may be treated with the methods herein include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, or Waldenstrom's macroglobulinemia.

The patient may be any animal (e.g., a mammal), Including, but not limited to, humans, non-human primates, rodents, and the like. Typically, the patient is human. The methods of treatment and medical uses of the surrogates of the invention or compounds or compositions comprising surrogates of the invention promote tissue regeneration. The term “tissue” refers to part of an organism consisting of a cell or an aggregate of cells, optionally having a similar structure, function and/or origin. Examples of tissues include but are not limited to: epithelial tissues, such as skin tissue, stomach lining, pancreatic lining, liver; connective tissues, such as inner layers of skin, tendons, ligaments, cartilage, bone, fat, hair, blood; muscle tissues; and nerve tissues, such as glial cells and neurons.

The compositions and method of the present invention may be combined with additional therapeutic agents. For example, when the disease, disorder or condition to be treated is a neoplastic disease (e.g. cancer) the methods of the present in Invention may be combined with IDconventional chemotherapeutic agents or other biological anti-cancer drugs such as checkpoint inhibitors (e.g. PD1 or PDL1 inhibitors) or therapeutic monoclonal antibodies (e.g Avastin, Herceptin).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571). Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatin1b; AN-152 (AEZS-108) doxorubicin linked to [D-Lys(6)]-LHRH; Braf Inhibitors, such as vemurafenib, dabrafenib, and LGX818; MEK Inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolmus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFa, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) Inhibitors, steroids, TNF antagonists (e.g., Remicade® and Enbrel®), interferon-β1a (Avonex®), and Interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an anti-CD93 ABD polypeptide or engineered cell may include, without limitation, Rituximab (marketed as MabThera or Rituxan), Alemtuzumab, Panitumumab, Ipilimumab (Yervoy), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), and atezolizumab (Tecentriq®, Genentech/Roche, South San Francisco CA). Additional examples of PD1 inhibitory antibodies include but are not limited to durvalumab (MED14736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

The invention now being fully described, it will be apparent to one of ordinary skill in the art that various changes and modifications can be made without departing from the spirit or scope of the invention.

### EXPERIMENTAL

### Context-Dependent Cytokine Adaptors

The tumor microenvironment supports cancer cell growth and promotes immune cell tolerance by inducing cell-surface and secreted factors which suppress immune activation. Central to this activity is the linkage between ligands and their receptors. Cytokine adaptors of the disclosure use a bi-specific binding protein to alter the specificity of receptors for their ligands, thereby converting suppressive factors into proinflammatory signals. Generally, cytokine adaptors consist of a cytokine receptor binding domain (for example a nanobody, scFv, dominant negative cytokine) and ligand (target protein) binding domain (nanobody, scFv, receptor extracellular domain). When two such molecules engage a suppressive ligand, they trigger cytokine receptor dimerization and downstream signaling. In this way, cytokine receptor dimerization and signaling depends on the presence of the suppressive ligand. Critically, the suppressive ligand must either be a dimer or have multiple binding proteins with non-overlapping epitopes so that binding of the adaptors can trigger dimerization and signaling of cytokine receptors. Described below is a specific example of a TGF-β-IL2R cytokine adaptor that converts suppressive TGF-β signaling into activating IL-2 signaling in lymphocytes.

TGF-β suppresses T cell proliferation and induces differentiation of regulatory T cells. In contrast, IL-2 is a lymphocyte activation signal which induces proliferation. Converting TGF-β based signaling to IL-2 based signaling has therapeutic value by enhancing local activation of T cells in tumors through the promotion of pro-inflammatory signaling.

In order to convert TGF-β based signaling to IL-2 based signaling, cytokine adaptors were developed. The cytokine adaptors comprised a target binding domain and a target signal activator domain, and can be two chain or single chain. The adaptors are listed in Table 1.

TABLE 1

| | | Target | | | |
|---|---|---|---|---|---|
| | receptor | protein | | | |
| | binding | binding | | | |
| Name | domain | domain | linker | | |
| CRG403* | IL- | scFvGC1008 | | | |
| SEQ ID | 2RβNb6 | | | | |
| NO: 1 | | | | | |
| CRG404 | γcNb6 | scFvGC1008 | | | |
| SEQ ID | | | | | |
| NO: 4 | | | | | |
| GA239 | γcNb6 | scFvGC1008 | SEQ ID | βNb6 | scFvGC1008 |
| | | | NO: 9 | | |
| GA242 | βNb6 | scFvGC1008 | SEQ ID | γcNb6 | scFvGC1008 |
| | | | NO: 8 | | |
| GA243 | βNb6 | scFvGC1008 | SEQ ID | γcNb6 | scFvGC1008 |
| | | | NO: 9 | | |
| GA244 | βNb6 | scFvGC1008 | SEQ ID | γcNb6 | scFvGC1008 |
| | | | NO: 10 | | |
| BS180⁺ | βNb6 | scFv9D7 | | | |
| SEQ ID | | | | | |
| NO: 20 | | | | | |
| BS181 | γcNb6 | scFv9D7 | | | |
| SEQ ID | | | | | |
| NO: 21 | | | | | |
| BS182^{#} | βNb6 | IL-10Rα | | | |
| SEQ ID | | | | | |
| NO: 16 | | | | | |
| BS183 | γcNb6 | IL-10Rα | | | |
| SEQ ID | | | | | |
| NO: 17 | | | | | |
| KH319^{@} | IL- | 22E11 | GGS | | |
| SEQ ID | 10RβNb6 | | | | |
| NO: 18 | | | | | |
| KH323 | 37D5 | monoIL- | GGS | | |
| SEQ ID | | 10DN | | | |
| NO: 19 | | | | | |

*administered as a pair with CRG404
⁺administered as a pair with BS181
^{#}administered as a pair with BS183
^{@}administered as a pair with KH323

Each of the single component adapters GA239, GA242, GA243 and GA244 comprise SEQ ID NO:1 and SEQ ID NO:4, separated by a linker and in the orientation as indicated in Table 1.

Sequences of the adaptor components are shown in Table 2.

TABLE 2

| Binding specificity | Designation | Sequence ID |
|---|---|---|
| Target binding domain TGFβ | scFv GC1008 | SEQ ID NO: 2 |
| Signal activator domain IL-2Rβ | βNb6 | SEQ ID NO: 3 |
| Signal activator domain IL-2Rγ | γcNb6 | SEQ ID NO: 5 |
| Target binding domain IL-10 | scFv9D7 | SEQ ID NO: 6 |
| Target binding domain IL-10 | IL-10Rα | SEQ ID NO: 7 |
| Target binding domain IL-23p19 | VHH 37D5 | SEQ ID NO: 12 |
| Target binding domain IL-23p40 | VHH 22E11 | SEQ ID NO: 13 |
| Signal activator domain IL-10Rα | monoIL-10DN | SEQ ID NO: 14 |
| Signal activator domain IL-10Rβ | IL-10RβNb6 | SEQ ID NO: 15 |

(SEQ ID NO: 1)
QVQLQESGGGSVQAGGSLRLSCAASSYTISSVCMGWFRQAPGKEREGVAGIAPDGSTGYGDSVKGRF
TISKDNAKNTLYLQMNSLKPEDTAMYYCAAASPGRCFLPRTALEPALYYNWGQGTQVTVSSQVQLVQS
GAEVKKPGSSVKVSCKASGYTFSSNVISWVRQAPGQGLEWMGGVIPIVDIANYAQRFKGRVTITADEST
STTYMELSSLRSEDTAVYYCASTIGLVLDAMDYWGQGTLVTVSSGGGGSGGGGSGGGGSALETVLTQ
SPGTLSLSPGERATLSCRASQSLGSSYLAWYQQKPGQAPRLLIYGASSRAPGIPDRFSGSGSGTDFTLTI
SRLEPEDFAVYYCQQYADSPITFGQGTRLEIKRHHHHHH
(SEQ ID NO: 2)
QVQLVQSGAEVKKPGSSVKVSCKASGYTFSSNVISWVRQAPGQGLEWMGGVIPIVDIANYAQRFKGRV
TITADESTSTTYMELSSLRSEDTAVYYCASTLGLVLDAMDYWGQGTLVTVSSGGGGSGGGGSGGGGSA
LETVLTQSPGTLSLSPGERATLSCRASQSLGSSYLAWYQQKPGQAPRLLIYGASSRAPGIPDRFSGSGS
GTDFTLTISRLEPEDFAVYYCQQYADSPITFGQGTRLEIKR.
(SEQ ID NO: 3)
QVQLQESGGGSVQAGGSLRLSCAASSYTISSVCMGWFRQAPGKEREGVAGIAPDGSTGYGDSVKGRF
TISKDNAKNTLYLQMNSLKPEDTAMYYCAAASPGRCFLPRTALEPALYYNWGQGTQVTVSS.
(SEQ ID NO: 4)
QVQLQESGGGSVQAGGSLRLSCAASGYTYRDYYMGWFRQAPGREREGVASIYTRGSREGSTRYSSSV
EGRFTITLDTAKNTLYLQMNSLKPEDTAMYYCAADDRTWLPRVOLGGPRENEYNYWGQGTQVTVSSQV
QLVQSGAEVKKPGSSVKVSCKASGYTFSSNVISWVRQAPGQGLEWMGGVIPIVDIANYAQRFKGRVTIT
ADESTSTTYMELSSLRSEDTAVYYCASTLGLVLDAMDYWGQGTLVTVSSGGGGSGGGGGGGGSALE
TVLTQSPGTLSLSPGERATLSCRASQSLGSSYLAWYQQKPGQAPRLLIYGASSRAPGIPDRFSGSGSGT
DFTLTISRLEPEDFAVYYCQQYADSPITFGQGTRLEIKRHHHHHH
(SEQ ID NO: 5)
QVQLQESGGGSVQAGGSLRLSCAASGYTYRDYYMGWFRQAPGREREGVASIYTRGSREGSTRYSSSV
EGRFTITLDTAKNTLYLQMNSLKPEDTAMYYCAADDRTWLPRVQLGGPRENEYNYWGQGTQVTVSS
SEQ ID NO: 6
QVNLLQSGAALVKPGASVKLSCKASGYTFTDFYIHWVKQSHGKSLEWIGYINPNSGYTNYNEKFKNKATL
TVDKSTSTGYMELSRLTSEDSANYSCTRGVPGNNWFPYWGQGTLVTVSSGGGGSGGGGSGGGGSAL
DTVLTQPPALTVSPGEKLTISCKASESVTSRMHWYQQKPGQQPKLLIYKASNLASGVPARFSGSGSGTD
FTLTIDPVEADDTAIYFCQQSWNGPLTFGAGTKLELKR
SEQ ID NO: 7
HGTELPSPPSVWFEAEFFHHILHWTPIPNQSESTCYEVALLRYGIESWNSISNCSQTLSYDLTAVTLDLYH
SNGYRARVRAVDGSRHSNWTVTNTRFSVDEVTLTVGSVNLEIHNGFILGKIQLPRPKMAPANDTYESIFS
HFREYEIAIRKVPGNFTFTHKKVKHENFSLLTSGEVGEFCVQVKPSVASRSNKGMWSKEECISLTRQYFT
VTN
SEQ ID NO: 8:
GGGGGGGGS
SEQ ID NO: 9:
GGGGGGGGSGGGGSGGGGS
SEQ ID NO: 10:
GGGGSGSGGGSGGGGSGSGGGGSGSGGGGS
There is no linker between domains for CRG403, CRG404, BS180, BS181,
BS182, BS183.
SEQ ID NO: 12
EVQLVESGGGLVQPGGSLALSCAASGFTLDYLAIGWFRQAPGKEREGVSCVSSSGQYTYYADSVKGRF
TISRDNAESTVYLQMNSLKPEDTAVYYCATDPECYRVRGYYNGEYDYWGQGTQVTVSS
SEQ ID NO: 13
EVQLVESGGGLVQAGGSLRLSCAASGRTFSWSAVGWFRQAPGKEREFVAAIRWSGGSPYYADSVKDR
FTISRDNAKNTVYLQMNSLRPEDTAVYLCGETSLFPTSRGSHYDTWGQGTQVTVSS
SEQ ID NO: 14
SPGQGTQSENSCTHFPGNLPAALRALRDAFSRVKTFFQMKDQLDNLLLKESLLEDFKGYLGCQALSEMI
QFYLEEVMPQAENQDPDIKAHVNSLGENLKTLRLRLRRCHRFLPCENGGGSGGKSKAVEQVKNAFNKL
QEKGIYKAMSEFDIFINYIEAYMTMKIRN
SEQ ID NO: 15
QVQLQESGGGSVQTGGSLRLSCAASQVNMNSVGWFRQAPGKAREGVATISPSGGATYYTDSVKGRFT
ISRDNAKNTVYLQMNSLNAEDTAMYYCAADGGRDWWLLRPQTFSYWGQGTQVTVSS
(BS182)
SEQ ID NO: 16
QVQLQESGGGSVQAGGSLRLSCAASSYTISSVCMGWFRQAPGKEREGVAGIAPDGSTGYGDSVKGRF
TISKDNAKNTLYLQMNSLKPEDTAMYYCAAASPGRCFLPRTALEPALYYNWGQGTQVTVSSGGGGSHG
TELPSPPSVWFEAEFFHHILHWTPIPNQSESTCYEVALLRYGIESWNSISNCSQTLSYDLTAVTLDLYHSN
GYRARVRAVDGSRHSNWTVTNTRFSVDEVTLTVGSVNLEIHNGFILGKIQLPRPKMAPANDTYESIFSHF
REYEIAIRKVPGNFTFTHKKVKHENFSLLTSGEVGEFCVQVKPSVASRSNKGMWSKEECISLTRQYFTVT
N
SEQ ID NO: 17 (BS183):
QVQLQESGGGSVQAGGSLALSCAASGYTYRDYYMGWFRQAPGREREGVASIYTRGSREGSTRYSSSV
EGRFTITLDTAKNTLYLQMNSLKPEDTAMYYCAADDRTWLPRVQLGGPRENEYNYWGQGTQVTVSSG
GGGSHGTELPSPPSVWFEAEFFHHILHWTPIPNQSESTCYEVALLRYGIESWNSISNCSQTLSYDLTAVT
LDLYHSNGYRARVRAVDGSRHSNWTVTNTRFSVDEVTLTVGSVNLEIHNGFILGKIQLPRPKMAPANDTY
ESIFSHFREYEIAIRKVPGNFTFTHKKVKHENFSLLTSGEVGEFCVQVKPSVASRSNKGMWSKEECISLTR
QYFTVTN
SEQ ID NO: 18 (KH319):
QVQLQESGGGSVQTGGSLRLSCAASQVNMNSVGWFRQAPGKAREGVATISPSGGATYYTDSVKGRFT
ISRDNAKNTVYLQMNSLNAEDTAMYYCAADGGRDWWLLRPQTFSYWGQGTQVTVSSGGSEVQLVESG
GGLVQAGGSLRLSCAASGRTFSWSAVGWFRQAPGKEREFVAAIRWSGGSPYYADSVKDRFTISRDNA
KNTVYLQMNSLRPEDTAVYLCGETSLFPTSRGSHYDTWGQGTQVTVSSGAPGS
SEQ ID NO: 19 (KH323):
EVQLVESGGGLVQPGGSLRLSCAASGFTLDYLAIGWFRQAPGKEREGVSCVSSSGQYTYYADSVKGRF
TISRDNAESTVYLQMNSLKPEDTAVYYCATDPECYRVRGYYNGEYDYWGQGTQVTVSSGGSSPGQGT
QSENSCTHFPGNLPAALRALRDAFSRVKTFFQMKDQLDNLLLKESLLEDFKGYLGCQALSEMIQFYLEEV
MPQAENQDPDIKAHVNSLGENLKTLRLRLRRCHRFLPCENGGGSGGKSKAVEQVKNAFNKLQEKGIYK
AMSEFDIFINYIEAYMTMKIRN

Cytokine adaptors are comprised of binding domains specific for an inhibitory cytokine (for example an scFv, VHH, or soluble receptor) linked to binding domains specific for stimulatory cytokine receptors (for example a VHH or scFv). Cytokine adaptors convert an inhibitory signal into a stimulatory signal by blocking the inhibitory cytokine from binding to its receptor and instead compelling dimerization of the stimulatory receptor. (PDB: 2PJY, 2B51).

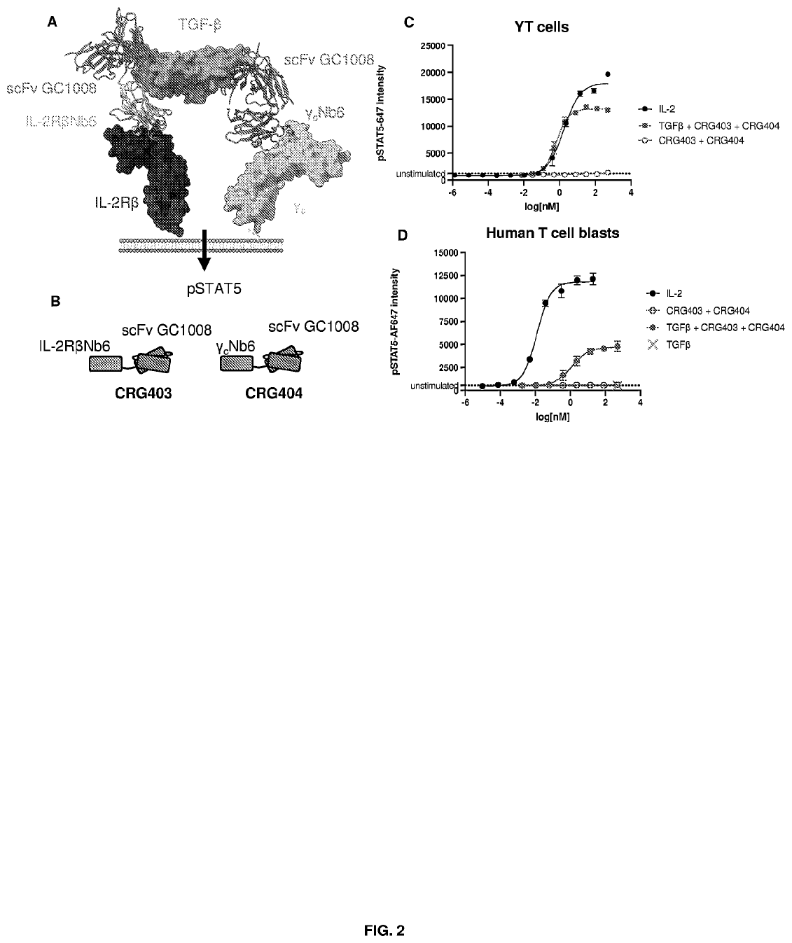

As shown in FIG. 2, cytokine adaptors CRG403 and CRG404 convert TGF-β stimulation into IL-2 signaling. (A) Model of cytokine adaptors, comprised of TGF-β and scFv structure (PBD: 4KV5) overlayed with structures of IL-2RβNb6 bound to IL-2Rβ (PDB: 7S2S) and γcNb6 bound to γc (PDB:7S2R). (B) Schematic illustrating the design of TGF-β adaptors CRG403 and CRG404.2 (C) Dose-response curves for phospho-STAT5 in YT cell line stimulated for 20 minutes with human IL-2 or TGF-β with equimolar CRG403 and CRG404. (D) Dose-response curves for phospho-STAT5 in human T cell blasts stimulated for 20 minutes with human IL-2 or TGF-β with equimolar CRG403 and CRG404.

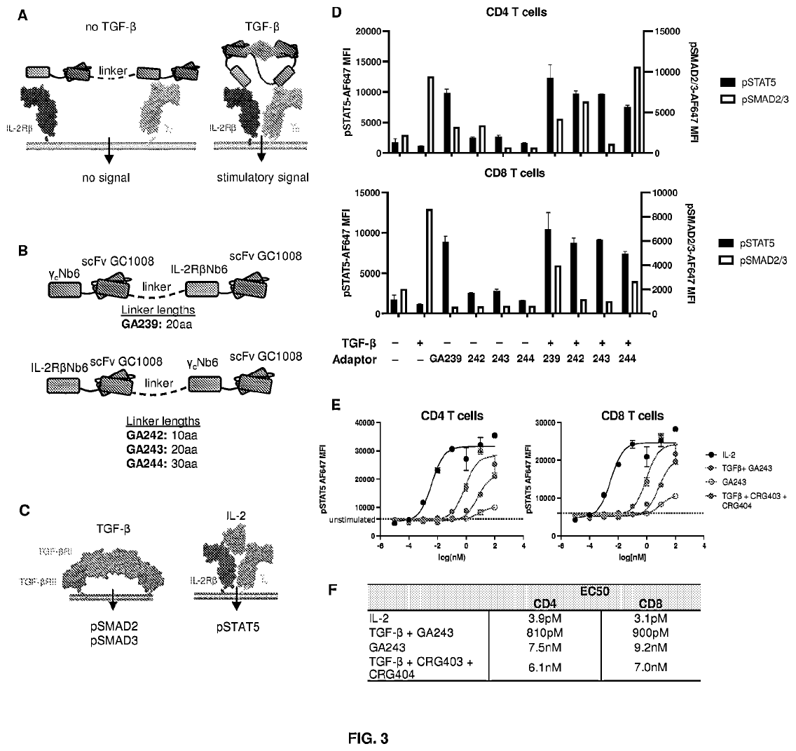

A single-component cytokine adaptor, GA243, modulates IL-2 receptor dimerization in human primary T cells. Shown in FIG. 3: (A) Model illustrating the rationale for designing single-component adaptors containing a flexible linker, such that receptor dimerization is compelled only in the presence of TGF-β. (B) Schematic illustrating the design of single-component cytokine adaptors GA239, GA242, GA243, and GA244. GA239 contains N-terminal γ_{c}Nb6 and a 20aa linker, while GA242, GA243, and GA244 feature a reversed orientation and linker lengths of 10aa, 20aa, and 30aa respectively. In the single-component design of the cytokine adaptor, γ_{c}Nb6 and IL-2RβNb6 are distant and unstructured in the absence of TGF-β but are brought close to compel receptor dimerization and signaling in the presence of TGF-β. (C) Schematic Illustrating downstream transcription factors activated by TGF-β signaling vs. IL-2 signaling. TGF-β receptor signaling leads to activation of pSMAD2 and pSMAD3 while IL-2 signaling leads to activation of pSTAT5. (D) Mean fluorescence intensity of phospho-STAT5 and phospho-SMAD2/phospho-SMAD3 in human CD4 and CD8 T cell blasts treated with 10 nM TGF-β+/−GA239, GA242, GA243, or GA244. (E) Dose-response curves for phospho-STAT5 in human CD4 and CD8 T cell blasts stimulated for 20 minutes with human IL-2, GA243, TGF-β with equimolar GA243, or TGF-β with equimolar CRG403 and CRG404. (F) EC50 values for phospho-STAT5 were calculated from sigmoidal dose-response curves.

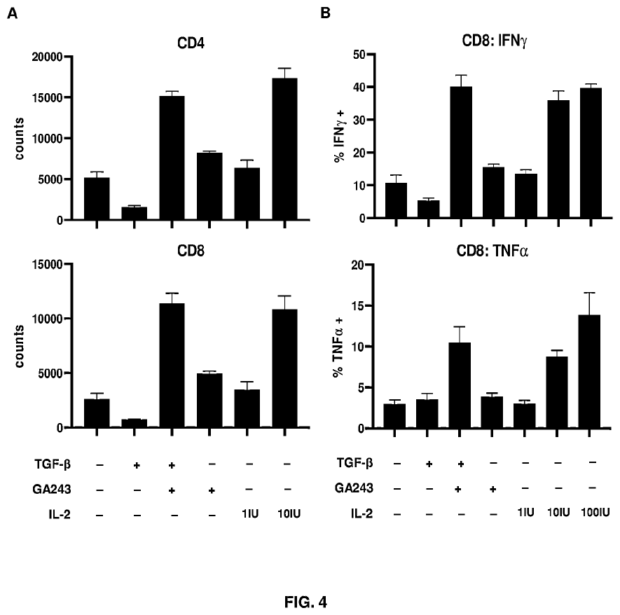

GA243 reverses T cell inhibition and promotes cytotoxicity in human primary T cells, as shown in FIG. 4. (A-B) Human T cell blasts were pre-activated with anti-CD3 and anti-CD28 for 48 hours, rested overnight, then cultured for 6 days +/−500 ng/mL TGF-β+/− equimolar GA243, or IL-2 (1, 10, or 1001U). On day 6, CD4 and CD8 T cell counts were quantified by flow cytometry (A), and CD8 T cells were treated with PMA, ionomycin, brefeldin A and monensin for 6 hours followed by intracellular cytokine staining for IFNγ and TNFα and analysis by flow cytometry (B).

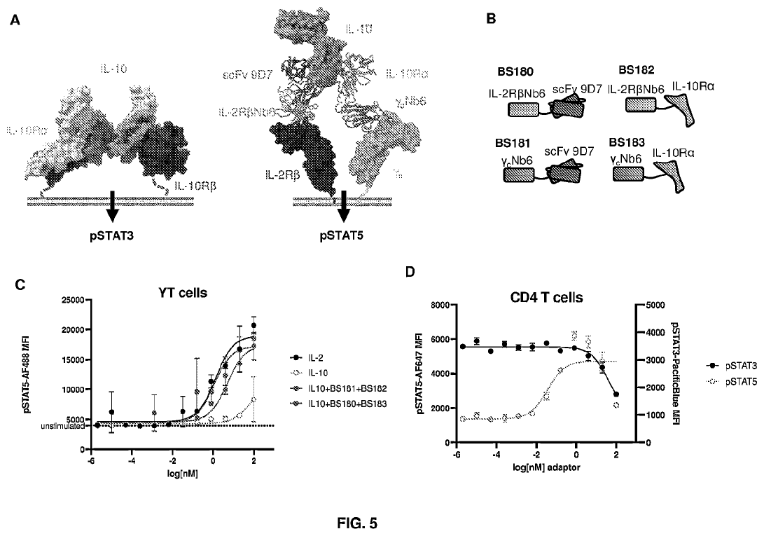

Cytokine adaptors BS180-BS183 convert IL-10 stimulation into IL-2 signaling, shown in FIG. 5. (A) Structure of IL-10 and its receptors (PDB: 6X93) and model of cytokine adaptors, comprised of structures of IL-10 and scFv 9D7 (PDB: 1LK3) overlayed with IL-10 and IL-10Rα (PDB: 6X93), IL-2RβNb6 bound to IL-2Rβ (PDB: 7S2S) and γcNb6 bound to γc (PDB:7S2R). (B) Schematic illustrating the design of IL-10 adaptors BS180, BS181, BS182, and BS183. (C) Dose-response curves for phospho-STAT5 in YT cell line stimulated for 20 minutes with human IL-2 or IL-10 with equimolar BS181 and BS182, or BS180 and BS183. (D) Dose-response curves for phospho-STAT5 and phospho-STAT3 in human CD4 T cell blasts stimulated for 20 minutes with 10 nM IL-10 and increasing concentrations of equimolar BS180 and BS183.

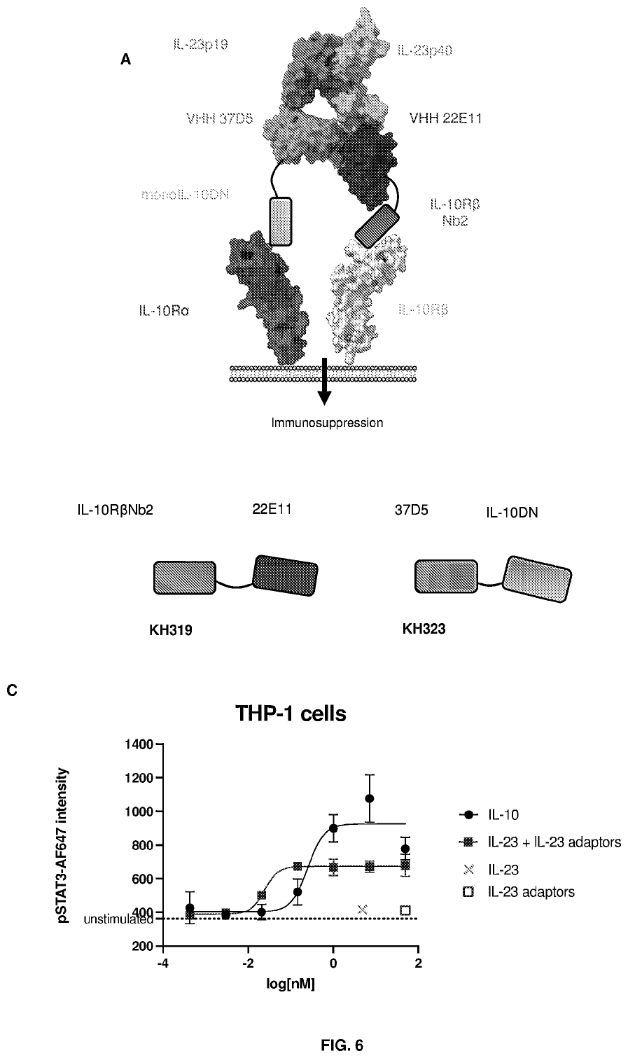

Cytokine adaptors can also convert proinflammatory cytokines into immunosuppressive cytokines. For example, KH319 and KH323 convert IL-23 into IL-10-like signaling, shown in FIG. 6 (A) Model of IL-23→IL-10 adaptors, comprised of IL-23p19 binder VHH 37D5 and IL-23p40 binder VHH 22E11 and IL-23p19 (PBD: 4GRW) overlayed with structures of IL-10 receptor complex (PDB: 6X93). (B) Schematic of IL-23-IL-10 cytokine adaptor design of molecules KH319 and KH323. KH319 is comprised of an IL-23p19 binding domain, VHH 37D5, linked to a monomeric dominant negative mutant of IL-10 (monoIL-10DN). KH323 is comprised of an IL-23p40 binding domain, VHH 22E11, linked to an IL-10Rβ binding module, IL-10RPNb2 (C) Dose-response curves for phospho-STAT3 in THP-1 cell line stimulated for 20 minutes with human IL-10 or IL-23 with equimolar IL-23 cytokine adaptors.

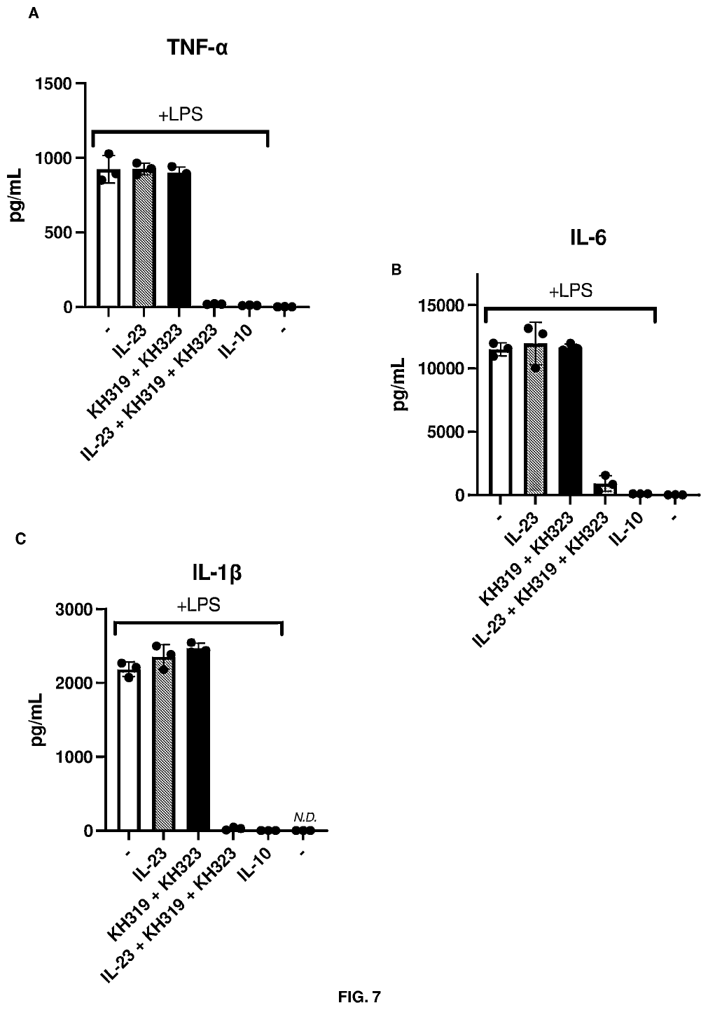

The IL-23 adaptors KH319 and KH323 reverse LPS-mediated inflammation in human peripheral mononuclear cells (PBMCs), as shown in FIG. 7. Human PBMCs were stimulated for 24 hours with 1 ng/mL LPS with or without the addition of 10 nM IL-23, KH319, KH323, or IL-10. The IL-23+KH319+KH323 condition suppressed LPS-mediated secretion of TNF-α (FIG. 7A), IL-6 (FIG. 7B), and IL-1β (FIG. 7C) equivalent to the suppression achieved with IL-10. These results show that IL-23 adaptors can inhibit inflammatory cytokine secretion in human PBMCs.

### Methods

Protein expression of TGFβ adaptors CRG403 and CRG404. Constructs comprising of an HA signal peptide, a N-terminal nanobody against IL-2Rβ or IL-2Rγ fused to anti-TGFβ scFv GC1008, and C-terminal 6×His tag were transiently expressed in Exp293 cells (Gibco). Cytokine Adaptors were purified by NiNTA affinity chromatography followed by S75 size-exclusion chromatography. Cytokine adaptors were then denatured and run through a SDS-PAGE gel to confirm purity (FIG. 7)

Protein expression of single-component TGFβ adaptors GA239, GA242, GA243, and GA244. Constructs comprising of an HA signal peptide, a N-terminal nanobody against IL-2Rγ (GA239) or IL-2Rβ (GA242, GA243, GA244) fused to anti-TGFβ scFv GC1008, followed by a linker of either 10 amino acids (GA242), 20 amino acids (GA239, 243), or 30 amino acids (GA244) linker, fused to a nanobody against or IL-2Rβ (GA239) IL-2Rγ (GA242, GA243, GA244) followed by anti-TGFβ scFv GC1008, and C-terminal 6×His tag were cloned into the pD649 expression vector. Protein was produced by transient transfection of Expi293F cells (Gibco). Cytokine Adaptors were purified by NiNTA affinity chromatography followed by S200 size-exclusion chromatography.

Protein expression of IL-10 adaptors BS180, BS181, BS182, and BS183. Constructs comprising of an HA signal peptide, a N-terminal nanobody against IL-2Rβ or IL-2Rγ fused to anti-IL-10 scFv 9D7 or receptor IL-10Rα, and C-terminal 6×His tag were cloned into the pD649 expression vector. Protein was produced by transient transfection of Expi293F cells (Gibco) using ExiFectamine 293 Transfection kit (Gibco) according to manufacturer protocols. Cytokine Adaptors were purified by NiNTA affinity chromatography followed by size-exclusion chromatography.

Protein expression of human IL-10. Constructs comprising of an HA signal peptide, human IL-10, and C-terminal 6×His tag were cloned into the pD649 expression vector. Protein was produced by transient transfection of Expi293F cells (Gibco) using ExiFectamine 293 Transfection kit (Gibco) according to manufacturer protocols. IL-10 was purified by NINTA affinity chromatography followed by size-exclusion chromatography.

pSTAT5 signaling assays in YT cells. To determine if cytokine adaptors can activate IL-2 based signaling, YT cells, a human NK cell line, were cultured in 100 μL complete RPMI and stimulated for 20 minutes+/−varying concentrations hTGFβ1 (R&D systems), +/− equimolar cytokine adaptors CRG403, CRG404 in a 96-well plate. For the IL-10 adaptors BS180, BS181, BS182, BS183, 2-3×10⁵ YT cells were cultured in 100 μL complete RPMI and stimulated for 20 minutes+/−titrations of IL-10+/− equimolar cytokine adaptors BS180, BS181, BS182, and BS183 in a 96-well plate. Cells were then fixed with 1.6% paraformaldehyde for 10 minutes at room temperature. Cells were permeabilized with 100% ice-cold methanol and stored at −20° C. prior to staining. Cells were washed twice with FACS buffer (PBS pH7.2, 2% FBS, 2 mM EDTA) and stained with 1:100 Alexa Fluor® 647 anti-STAT5 pY694 (BD) for 1 hour at room temperature. Mean fluorescence intensity (MFI) was measured using a CytoFLEX flow cytometer (Beckman Coulter). Dose-response curves were generated using Prism (GraphPad).

pSMAD2/pSMAD3 and pSTAT5 signaling assays in human T cells. Human peripheral blood monocytes (PBMCs) from healthy human donors cultured for 48 hours in complete RPMI supplemented with 5 μg/mL anti-CD28 (BioLegend) in 6-well plates pre-coated with 2.5 μg/mL anti-CD3 (OKT3, BioLegend). Cells were washed twice with PBS and rested overnight in complete RPMI. Cells were stained with human TruStain FcXN (Biolegend), Pacific Bluem anti-human CD4 antibody, OKT4 (Biolegend), and Brilliant Violet 605™, anti-human CD8a (Biolegend) in FACS buffer for 15 minutes at 4° C. For signaling assays, 2×10⁵ cells were resuspended in 100 μL complete RPMI and stimulated for 20 minutes+/− varying concentrations hTGFβ1 (R&D systems)+/−equimolar CRG403, CRG404, GA239, GA242, GA243, or GA244 in a 96-well plate. Fixed and permeabilized cells were stained with 1:100 Alexa Fluor®647 anti-STAT5 pY694 (BD), 1:100 Alexa Fluor® 488 anti-STAT5 pY694 (BD), or 1:100 Alexa Fluor® 647 anti-Smad2 (pS465/pS467)/Smad3 (pS423/pS425) (BD) for 1 hour at room temperature. Mean fluorescence intensity (MFI) was measured using a CytoFLEX flow cytometer (Beckman Coulter).

pSTAT5 and pSTAT3 signaling assay in human T cells. Human PBMCs were prepared as described above and prepared for signaling assays as described above. 2×10⁵cells were cultured in 100 μL complete RPMI and stimulated for 20 minutes with 10 nM IL-10 and varying concentrations of BS180 and BS183. Cells were fixed and permeabilized as described above, then stained with 1:100 Alexa Fluor®647 anti-STAT5 pY694 (BD) and 1:100 Pacific Blue™ anti-STAT3 (pY705) (BD) for 1 hour at room temperature. Mean fluorescence intensity (MFI) was measured using a CytoFLEX flow cytometer (Beckman Coulter). Dose-response curves were generated using Prism (GraphPad).

Proliferation assay in human T cells. To determine the effects of the TGFβ adaptors on human T cell proliferation and activation, human PBMCs were isolated from healthy human donors and cultured for 48 hours in complete RPMI supplemented with 5 μg/mL anti-CD28 (BioLegend) in 6-well plates pre-coated with 2.5 μg/mL anti-CD3 (OKT3, BioLegend). Human T cell blasts were washed twice with PBS (GIBCO), rested overnight in RPMI complete, then cultured for 6 days in RPMI complete+/−500 ng/mL hTGFβ1 (R&D systems)+/−equimolar GA243, or IL-2 (1, 10, or 100 IU). CD4 and CD8 T cell counts were quantified using a CytoFLEX flow cytometer (Beckman Coulter). For intracellular cytokine staining, cells were treated with 50 ng/mL phorbol 12-myristate 13-acetate (PMA) (Sigma) and 1 μg/mL ionomycin (Sigma), as well as GolgiPlug (BD) and Golgistop (BD) for 6 hours, fixed and permeabilized as per BD protocol, and stained with 1:100 Alexa Fluor® anti-human IFNγ antibody (4SB3, Biolegend) and PE-Cy7 anti-human TNF (mAb11, BD Biosciences) for 1 hour at room temperature. Fluorescence intensity was measured a CytoFLEX flow cytometer (Beckman Coulter) and analyzed using FlowJo (BD).

### REFERENCES

Yodoi, J., Teshigawara, K., Nikaido, T., Fukui, K., Noma, T., Honjo, T., Takigawa, M., Sasaki, M., Minato, N., Tsudo, M., et al. (1985). TCGF (IL 2)-receptor inducing factor(s). I. Regulation of IL 2 receptor on a natural killer-like cell line (YT cells). J Immunol 134, 1623-1630.

1. A cytokine adaptor, the cytokine adaptor comprising:
i) a target binding domain that specifically binds to a target protein, wherein the target protein forms a homo or heterodimer,
ii) an optional first linker, and
iii) a target signal activator binding domain that specifically binds to one component of a two component signaling complex.

2. (canceled)

3. The cytokine adaptor of claim 1, wherein the target protein is a tumor-associated protein, optionally selected from transforming growth factor beta (TGF-β), IL-10, IL-23, IL-17, vascular endothelial growth factor (VEGF), programmed death-ligand 1 (PDL1), human epidermal growth factor 2 (HER2), epidermal growth factor receptor (EGFR), fibroblast activation protein (FAP), tumor-associated calcium signal transducer 2 (Trop2), epithelial cell adhesion molecule (EPCAM), prostate-specific membrane antigen (PSMA), and arginase 2 (ARG2).

4-6. (canceled)

7. The cytokine adaptor of claim 1, wherein the target protein is a proinflammatory cytokine, optionally selected from IL-23, IL-17A, IL-17B, IL-17C, IL-17C, IL-17D, IL-17E, IL-17F, IL-1β, IL-6, and TNF-α.

8. (canceled)

9. The cytokine adaptor of claim 1, wherein the target signal activator domain specifically binds to one component of a two component signaling complex selected binding from IL-2 receptor beta (IL-2Rβ), IL-2R gamma (IL-2Rγ), interferon-α/β receptor 1 (IFNAR1), interferon-α/β receptor 2 (IFNAR2), and IL-10Rα/β.

10. The cytokine adaptor of claim 1, wherein the first linker is 10 or more, 20 or more, or 30 or more amino acids in length.

11. A tandem adaptor comprising two cytokine adaptors of claim 1, joined by a second linker.

12. The cytokine adaptor of claim 1, wherein the target binding domain is a single-chain variable fragment (scFv) or an immunoglobulin single variable domain (ISV).

13. The cytokine adaptor of claim 1, wherein the target signal activator binding domain is a scFv or an ISV.

14. (canceled)

15. The cytokine adaptor of claim 1, comprising the sequence set forth in any of SEQ ID NO:1, SEQ ID NO:4, SEQ ID NO: 16, SEQ ID NO:17, SEQ ID NO:18, SEQ ID NO:19, SEQ ID NO:20, or SEQ ID NO:21

16. A method of treating cancer, the method comprising:
administering an effective dose of two different cytokine adaptors according to claim 1, wherein a first cytokine adaptor comprises the target signal activator binding domain that specifically binds to one component of the two component signaling complex and a second cytokine adaptor comprises the target signal activator binding domain that specifically binds to one component of the two component signaling complex that is different than the first cytokine adaptor.

17. (canceled)

18. A method of treating an inflammatory disease, the method comprising:
administering an effective dose of two different cytokine adaptors according to claim 1, wherein a first cytokine adaptor comprises the target signal activator binding domain that specifically binds to one component of the two component signaling complex and a second cytokine adaptor comprises the target signal activator binding domain that specifically binds to one component of the two component signaling complex that is different than the first cytokine adaptor.

19-21. (canceled)

22. The method of claim 18, wherein the target binding domain of the first and second cytokine adaptor binds to two different target proteins, optionally selected from IL-2Rα and IL-2Rγ; IFNAR1 and IFNAR2; and 10Rα and IL-10Rβ.

23-29. (canceled)

30. A polynucleotide encoding a cytokine adaptor of claim 1.

31. A vector comprising a polynucleotide of claim 30.

32. A cell comprising a polynucleotide of claim 30.

33. A method of producing a cytokine adaptor, the method comprising culturing the cell of claim 32 to express the encoded cytokine adapter protein.
